(19) United States
(12) Patent Application Publication
(10) Pub. No.: US 2012/0202891 A1
(43) Pub. Date: Aug. 9, 2012

(54) Cannabinoid-Containing Compositions and Methods for Their Use

(75) Inventors: Audra L. Stinchcomb, Lexington, KY (US); Stan Lee Banks, Frankfort, KY (US)

(73) Assignee: UNIVERSITY OF KENTUCKY RESEARCH FOUNDATION, Lexington, KY (US)

(21) Appl. No.: 13/318,208

(22) PCT Filed: Apr. 29, 2009
(86) PCT No.: PCT/US09/42124

§ 371 Date: Apr. 18, 2012

(51) Int. Cl. A61K 31/05; A61P 19/02

(52) U.S. Cl. 514/733

(57) ABSTRACT

This invention relates to cannabinoid-containing compositions, particularly cannabinoid-containing gel formulations and methods for the treatment of traumatic injury, e.g., strains, sprains and contusions, and disease conditions, e.g., arthritis, particularly osteoarthritis. The methods involve topically applying a cannabinoid or a cannabinoid-containing composition to a subject's skin near, or distant from, the area of the injury or the area affected by the disease condition, e.g., an arthritic joint. The cannabinoid-containing composition is preferably a pharmaceutically acceptable gel containing a therapeutically effective amount of a cannabinoid sufficient to alleviate the symptoms associate with the injury or disease condition.

This application is a national stage of PCT International Application No. PCT/US2009/042124, filed Apr. 29, 2009, the entire disclosure of which is herein expressly incorporated by reference.

### TECHNICAL FIELD

The present invention generally relates to the transdermal delivery of cannabinoids. More particularly, the present invention relates to methods and materials for transdermally delivering cannabinoids, particularly cannabidiol, to treat symptoms, e.g., inflammation, pain and discomfort, associated with or caused by traumatic injury or disease conditions, e.g., strains, sprains, contusions and arthritis, particularly osteoarthritis.

### BACKGROUND OF THE INVENTION

Arthritis is classified as one of the rheumatic diseases, which are related in that they have a tendency to affect the joints, muscles, ligaments, cartilage, and tendons, and many have the potential to affect other internal body areas. The cause of arthritis ranges from wear and tear and traumatic injury of cartilage (contributing to, e.g., osteoarthritis) to inflammation due to metabolic abnormalities (such as gout and pseudogout), hereditary, infections and overactive immune systems (associated with rheumatoid arthritis and systemic lupus erythematosus).

An estimated 46 million adults in the United States have self-reported and doctor diagnosed forms of arthritis and the problem is becoming an even bigger concern as the baby boomers age. Twenty eight million adults have the most common forms of arthritis: osteoarthritis and rheumatoid arthritis (Lawrence et al., (2008) Arthritis Rheum 58:26-35). According to the US Census as of December of 2001, the world population of people of 65 and older is increasing at a rate of 800,000/month. The United States is ranked 32nd with 13% of the current population over the age of 65. It is estimated that from the year 2000 to 2030 the number of people over 65 will be increased by 102%. Osteoarthritis is estimated to cost the US approximately 3.4-13.2 billion dollars a year including medical costs plus lost productivity (Leigh et al., (2001) J Rheumatol 28: 1647-1654). This debilitating disease condition is often untreatable, leading to limited performance of daily tasks and lower quality of life. No matter which form of arthritis is involved, pain is a constant accompanying factor in every case. Arthritis and its joint pain can be manifested as hip pain, knee pain, hand pain, or wrist pain, as well as joint pain elsewhere in the body.

The primary treatments for osteoarthritis pain are non-steroidal anti-inflammatory drugs (NSAIDs), administered either orally or topically, which have been associated with toxic side effects due to their role with inhibition of cyclo-oxygenase (COX) enzymes, particularly COX-2. Transdermally delivered opioids, such as fentanyl, provide pain relief for arthritis patients (Langford et al., (2006) Arthritis Rheum 54: 1829-1837). Intraarticular injections of glucocorticoid and hyaluronic acid (Gerwin et al., (2006) Adv Drug Deliv Rev 58: 226-242) are also available.

Cannabinoid receptors CB1 and CB2 are primarily found in the brain (nervous system) and immune system, respectively. Both CB receptors play a role in pain, however, CB1 receptor activation tends to be associated with psychoactive side effects whereas CB2 receptor activation is not (Clayton et al., (2002) Pain 96: 253-260). CB2 activation produced analgesia and reduced inflammation in rats (Clayton et al., 2002). Cannabinoids like Δ9-tetrahydrocannabinol (THC) work at either CB1 or CB2 as agonists. Some cannabinoids, such as cannabidiol (CBD), have little binding affinity for CB1 and CB2 receptors leaving their role in receptor activation or deactivation not completely understood. Others have suggested that CBD actions are actually mediated via a transient receptor potential vanilloid type 1 (TRPV1) (Costa et al., (2004b) Br J Pharmacol 143: 247-250).

When THC has been administered orally, side effects and lack of positive response lead to patients discontinuing in the trial (Attal et al. (2003) Eur J Pain 8: 173-177; Marlan Jr et al. (2003) Curr Opin Pharmacol 3: 62-67). The lack of positive response is often due to peak plasma level-related adverse effects. Side effects occur when effective/higher doses of the THC were administered to the point patients could no longer function in daily tasks (Attal et al. (2003) Eur J Pain 8: 173-177; Marlan Jr et al. (2003) Curr Opin Pharmacol 3: 62-67. CBD, while structurally similar to THC (FIG. 1), is a non-psychoactive component of marijuana. CBD has been examined for its therapeutic potential for neuropathic pain, cancer pain, multiple sclerosis, and inflammation (Burstein and Zurier (2009) AAPS J. Epub ahead of press. PMID: 19199042). CBD is more potent than aspirin (360x) and THC (590x).

Canabidiol (“CBD”) is the major nonpsychoactive component of cannabis. Malfait et al. (2000) (Proc. Natl. Acad. Sci. 97(17):9561-9566) explored the therapeutic potential of intraperitoneally (ip) and orally administered CBD in a murine model for rheumatoid arthritis, wherein mice were administered type II collagen in complete freund's adjuvant (CIA) (Courtenay et al. (1980) Nature (London) 283:666-668). Malfait et al. report that CBD had a beneficial therapeutic action on established CIA when administered systemically. Rats, which had received a complete Freund's adjuvant (CFA) intraplantar injection, were administered 20 mg/kg of CBD orally for 7 consecutive days (Costa et al. (2007) Eur J Pharmacol 556: 75-83). Results showed a 50% decrease in thermal and mechanical withdrawal latency and that repeated dosing was needed to decrease pain.

### SUMMARY OF THE INVENTION

Cannabinoids have been proven effective in relief of inflammatory pain. Topical application and transdermal delivery of cannabinoids, particularly CBD, allows for a more controlled drug delivery rate. Delivery of pain controlling drugs through the skin allows for targeting specific sites and joints for treatment. An increase in the duration of the effect of such drugs would significantly improve clinical efficacy. The results presented herein demonstrate of the in vivo effects of transdermal delivery of a cannabinoid-containing composition to CFA mono-arthritic knee joints in Sprague-Dawley rats. Our results demonstrate that transdermal delivery of the cannabinoid-containing composition, particularly a CBD-containing gel, decreased inflammation and pain without inducing abnormal behavior or other adverse effects.

The present invention relates to methods for alleviating the symptoms associated with arthritis, particularly osteoarthritis. The methods involve providing a cannabinoid-containing composition suitable for topical application and delivering the cannabinoid to a subject in need thereof. The cannabinoid may be one selected from the group consisting of cannabinol, cannabidiol, nabilone, levonantradol, (−)-HU-210, (+)-HU-210, 11-hydroxy-Δ⁹-THC, Δ⁸-THC-11-oic acid, CP 55,940, R(+)-WIN 55, 212-2 and Δ⁹-THC. Preferably the cannabinoid is cannabidiol and the cannabinoid containing composition is a pharmaceutically acceptable gel.

### BRIEF DESCRIPTION OF THE DRAWINGS

FIG. 1 depicts the structure of cannabidiol.

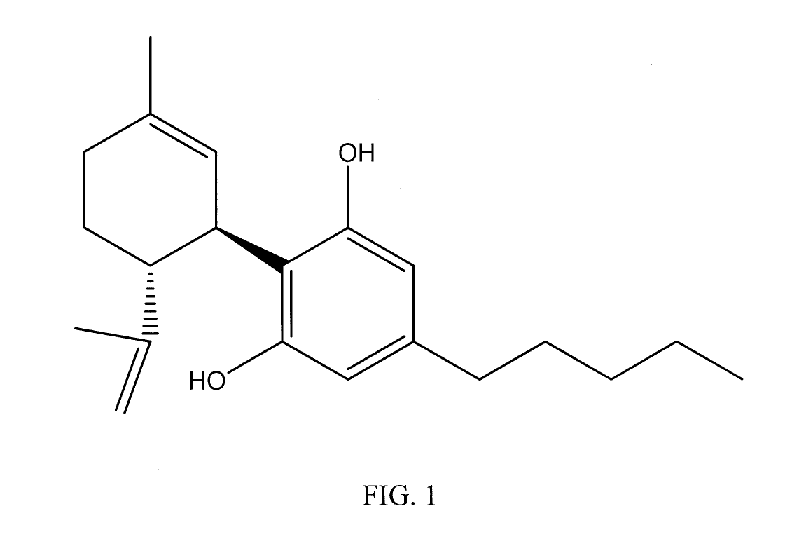

FIG. 2 A-B compares the paw withdrawal latency from (A) CFA induced rats treated with CBD and (B) normal rats treated with CBD. Data are represented as mean±SD. In FIG. 1A, a significant improvement (p<0.05) was observed in rats receiving 6.2 mg/d (9.2±0.7) and 62.3 mg/d (9.1±0.2) treatments as compared to rats receiving the vehicle gel (7.1±0.6), 0.6 mg/d (7.8±0.5), and 3.1 mg/d (7.6±0.8) treatments (normal+vehicle [], n=4; 0.6 mg [x], n=5; 3.1 mg [▴], n=5; 6.2 mg [▪], n=5; 62.3 mg [♦], n=3, CFA+vehicle [ [symbol] , n=11). In FIG. 1B, the PWL in normal animals was not affected by any CBD dose (normal+vehicle [], n=4; 0.6 mg [x], n=5; 3.1 mg [▴], n=5; 6.2 mg [▪], n=5).

FIG. 3 A-F compares the exploratory activities in normal control rats treated with different doses of CBD: (A) rearing events, (B) rearing time, (C) total beams broken, (D) distance traveled, (E) active time, (F) resting time [light gray bar=pre-drug, black bar=post-drug]. Data are represented as mean±SD. (0 mg, n=4; 0.6 mg, n=6; 3.1 mg, n=5; 6.2 mg, n=3; 62.3 mg, n=1). No significant differences (p>0.05) were determined among the different dose treatment groups for the six parameters.

### DETAILED DESCRIPTION OF THE INVENTION

The methods of the present invention involve applying a cannabinoid or cannabinoid containing composition to the skin of a subject in need thereof to transdermally or topically deliver a therapeutically effective amount of cannabinoid sufficient to alleviate the symptoms of osteoarthritis. The cannabinoid composition includes at least one cannabinoid selected from the group consisting of cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55, 212-2 and Δ⁹-THC.

The present invention overcomes the problems associated with existing drug delivery systems by delivering cannabinoids transdermally or topically. Preferably, the cannabinoids are delivered transdermally or topically via a pharmaceutically acceptable cannabinoid-containing composition, e.g., a cannabinoid-containing gel, to reduce harmful side effects and avoid gastrointestinal (first-pass) metabolism of the drug by the patient.

A first aspect of the invention provides a method for relieving symptoms associated with osteoarthritis in a subject, particularly a mammalian subject, wherein the symptoms of osteoarthritis include at least one of inflammation, pain or discomfort. The method comprises the steps of selecting at least one cannabinoid from the group consisting of cannabinol, cannabidiol, nabilone, levonantradol, (−)-HU-210, (+)-HU-210, 11-hydroxy-Δ⁹-THC, Δ⁸-THC-11-oic acid, CP 55,940, R(+)-WIN 55, 212-2, Δ⁹-THC, and prodrugs or any of the foregoing and applying the cannabinoid to the skin of a subject in need thereof in an amount and for a time sufficient to alleviate the symptoms of osteoarthritis.

A second aspect of the invention provides a viscous flowable pharmaceutically acceptable gel or gel-like composition comprised of a cannabinoid (such as cannabidiol), one or more penetration enhancers, a gelling agent and, optionally, a neutralizing agent. The cannabinoid with or without the penetration enhancer may be suspended within the gel or may be dissolved within the gel. The cannabinoid may be selected from the group consisting of cannabinol, cannabidiol, nabilone, levonantradol, (−)-HU-210, (+)-HU-210, 11-hydroxy-Δ⁹-THC, Δ⁸-THC-11-oic acid, CP 55,940, R(+)-WIN 55, 212-2 and Δ⁹-THC.

In one embodiment, compositions disclosed herein comprise cannabidiol in a total amount by weight of the composition of about 0.1% to about 95%. For example, the amount of cannabidiol by weight of the composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13% about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95%.

Illustratively, the compositions disclosed herein may comprise a total amount of cannabidiol by weight of about 1% to about 10%; about 2% to about 10%; about 3% to about 10%; about 4% to about 10%; about 5% to about 10%; about 6% to about 10%; about 7% to about 10%; about 8% to about 10%; about 9% to about 10%; about 1% to about 9%; about 2% to about 9%; about 3% to about 9%; about 4% to about 9%; about 5% to about 9%; about 6% to about 9%; about 7% to about 9%; about 8% to about 9%; about 1% to about 8%; about 2% to about 8%; about 3% to about 8%; about 4% to about 8%; about 5% to about 8%; about 6% to about 8%; about 7% to about 8%; about 1% to about 7%; about 2% to about 7%; about 3% to about 7%; about 4% to about 7%; about 5% to about 7%; about 6% to about 7%; about 1% to about 6%; about 2% to about 6%; about 3% to about 6%; about 4% to about 6%; about 5% to about 6%; about 1% to about 5%; about 2% to about 5%; about 3% to about 5%; about 4% to about 5%; about 1% to about 4%; about 2% to about 4%; about 3% to about 4%; about 1% to about 3%; about 2% to about 3%; or about 1% to about 2%.

The term “therapeutically effective amount” or “therapeutically and/or prophylactically effective amount” as used herein refers to an amount of compound or agent that is sufficient to elicit the required or desired therapeutic and/or prophylactic response, as the particular treatment context may require.

It will be understood that a therapeutically and/or prophylactically effective amount of a drug for a subject is dependent inter alia on the body weight of the subject as well as other factors known to a person of ordinary skill in the art. A “subject” herein to which a therapeutic agent or composition thereof can be administered includes mammals such as a human subject of either sex and of any age, and also includes any nonhuman animal, particularly a domestic or companion animal, illustratively a cat, dog or a horse as well as laboratory animals such as guinea pigs.

The terms “treat”, “treated”, “treating” and “treatment” are to be broadly understood as referring to any response to, or anticipation of, a medical condition in a mammal, particularly a human, and includes but is not limited to:
preventing the medical condition from occurring in a subject, which may or may not be predisposed to the condition, but has not yet been diagnosed with the condition and, accordingly, the treatment constitutes prophylactic treatment for the medical condition;
inhibiting the medical condition, e.g., arresting, slowing or delaying the onset, development or progression of the medical condition; or
relieving the medical condition, e.g., causing regression of the medical condition or reducing the symptoms of the medical condition.

In one embodiment, a therapeutically effective amount of cannabidiol is administered to treat a medical condition selected from the group consisting of: nausea, emesis, pain, wasting syndrome, HIV-wasting, chemotherapy induced nausea and vomiting, alcohol use disorders, dystonia, multiple sclerosis, inflammatory bowel disorders, arthritis, dermatitis, Rheumatoid arthritis, systemic lupus erythematosus, anti-inflammatory, anti-convulsant, anti-psychotic, antioxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, osteoarthritis, rheumatoid arthritis, synovitis, juvenile rheumatoid arthritis and inhibition of hair growth.

In a further embodiment the cannabidiol gels described herein are suitable for use for the relief of the pain of osteoarthritis of the joints, such as the hands, feet, ankles, wrists, shoulders, back, elbows and knees as well as the acute pain due to minor sprains, strains and contusions.

In one embodiment, the pharmaceutical composition containing cannabidiol is administered once daily to a subject in need thereof. In a further embodiment, the pharmaceutical composition containing cannabidiol or a cannabidiol prodrug is administered twice daily to a subject in need thereof. In a further embodiment, the pharmaceutical composition is administered more than twice daily, such as three, four, five, six, seven or eight times daily.

Pharmaceutical Excipients

The pharmaceutical compositions described herein can, if desired, include one or more pharmaceutically acceptable excipients. The term “excipient” herein means any substance, not itself a therapeutic agent, which may be used as a carrier or vehicle for delivery of a therapeutic agent to a subject or combined with a therapeutic agent (e.g., to create a pharmaceutical composition) to improve its handling or storage properties or to permit or facilitate formation of a dose unit of the composition. Excipients include, by way of illustration and not limitation, binders, disintegrants, taste enhancers, solvents, thickening or gelling agents (and any neutralizing agents, if necessary), penetration enhancers, solubilizing agents wetting agents, antioxidants, lubricants, emollients, substances added to mask or counteract a disagreeable odor, fragrances or taste, and substances added to improve appearance or texture of the composition. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of the cannabidiol or cannabidiol prodrug.

In one embodiment, the cannabidiol can be combined with one or more penetration enhancing agent for transdermal or topical delivery. A penetration enhancer is an excipient that aids in the diffusion of the active through the stratum corneum. Penetration enhancers are also known as permeation enhancers, accelerants, adjuvants or sorption promoters. A suitable penetration enhancer for use in the compositions and methods described herein should: (i) highly potent, with a specific mechanism of action; (ii) exhibit a rapid onset upon administration; (iii) have a predictable duration of action; (iv) have only non-permanent or reversible effects on the skin; (v) the enhancer should be chemically stable; (vi) have no or minimal pharmacological effects; (vii) be physically and chemically compatible with other formulation components; (viii) be odorless; (ix) be colorless; (x) be hypoallergenic; (xi) be non-irritating; (xii) be non-phototoxic; (xiii) be non-comedogenic; (xiv) have a solubility parameter approximating that of the skin (10.5 cal/cm3); (xv) be readily available; (xvi) inexpensive; and (xvii) be able to formulated in pharmaceutical compositions for topical or transdermal delivery of an active pharmaceutical agent.

Several classes of chemical compounds, with various mechanisms of action, can be used as penetration enhancers. Set forth below are non-limiting examples of penetration enhancing agents. Sulfoxides, such as dimethylsulfoxide and decylmethylsulfoxide can be used as penetration enhancing agents. Dimethylsulfoxide enhances penetration in part by increasing lipid fluidity and promoting drug partitioning. In contrast, decylmethylsulfoxide enhances penetration by reacting with proteins in the skin that change the conformation of the proteins, which results in the creation of aqueous channels.

Another class of a penetration enhancers are alkanones, such as N-heptane, N-octane, N-nonane, N-decane, N-undecane, N-dodecane, N-tridecane, N-tetradecane and N-hexadecane. Alkanones are thought to enhance the penetration of an active agent by altering the stratum corneum. A further class of penetration enhancers are alkanol alcohols, such as ethanol, propanol, butanol, 2-butanol, pentanol, 2-pentanol, hexanol, octanol, nonanol, decanol and benzyl alcohol. Lower molecular weight alkanol alcohols, i.e., those with 6 or less carbons, may enhance penetration in part by acting as solubilizing agents, while more hydrophobic alcohols may increase diffusion by extracting lipids from the stratum corneum. A further class of penetration enhancers are fatty alcohols, such as oleyl alcohol, caprylic alcohol, decyl alcohol, lauryl alcohol, 2-lauryl alcohol, myristyl alcohol, cetyl alcohol, stearyl alcohol, oleyl alcohol, linoleyl alcohol and linolenyl alcohol. Polyols, including propylene glycol, polyethylene glycol, ethylene glycol, diethylene glycol, triethylene glycol, dipropylene glycol, glycerol, propanediol, butanediol, pentanediol, hexanetriol, propylene glycol monolaurate and diethylene glycol monomethyl ether (transcutol) can also enhance penetration. Some polyols, such as propylene glycol may function as a penetration enhancer by solvating alpha-kertin and occupying hydrogen bonding sites, thereby reducing the amount of active-tissue binding.

Another class of penetration enhancers are amides, including urea, dimethylacetamide, diethyltoluamide, dimethylormamide, dimethyloctamide, dimethyldecamide and biodegradable cyclic urea (e.g., 1-alkyl-4-imidazolin-2-one). Amides have various mechanisms of enhancing penetration. For example, some amides, such as urea increase the hydration of the stratum corneum, act as a keratolytic and create hydrophilic diffusion channels. In contrast, other amides, such as dimethylacetamide and dimethylormamide, increase the partition to keratin at low concentrations, while increasing lipid fluidity and disrupting lipid packaging at higher concentrations. Another class of penetration enhancing agents are pyrrolidone derivatives, such as 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-methyl-pyrrolidone, N-cyclohexylpyrrolidone, N-dimethylaminopropyl-pyrrolidone, N-cocoalkypyrrolidone and N-tallowalkypyrrolidone, as well as biodegradable pyrrolidone derivatives, including fatty acid esters of N-(2-hydroxyethyl)-2-pyrrolidone. In part, pyrrolidone derivatives enhance penetration through interactions with the keratin in the stratum corneum and lipids in the skin structure. An additional class of penetration enhancers are cyclic amides, including 1-dodecylazacycloheptane-2-one (“Azone”), 1-geranylazacycloheptan-2-one, 1-farnesylazacycloheptan-2-one, 1-geranylgeranylazacycloheptan-2-one, 1-(3,7-dimethyloctyl)-azacycloheptan-2-one, 1-(3,7,11-trimethyldodecyl)azacyclohaptan-2-one, 1-geranylazacyclohexane-2-one, 1-geranylazacyclopentan-2,5-dione and 1-farnesylazacyclopentan-2-one. Cyclic amides, such as Azone, enhance the penetration of active agents in part by affecting the stratum corneum's lipid structure, increasing partitioning and increasing membrane fluidity. Additional classes of penetration enhancers include diethanolamine, triethanolamine and hexamethylenlauramide and its derivatives.

Additional penetration enhancers include linear fatty acids, such as octanoic acid, linoleic acid, valeric acid, heptanoic acid, pelagonic acid, caproic acid, capric acid, lauric acid, myristric acid, stearic acid, oleic acid and caprylic acid. Linear fatty acids enhance penetration in part via selective perturbation of the intercellular lipid bilayers. In addition, some linear fatty acids, such as oleic acid, enhance penetration by decreasing the phase transition temperatures of the lipid, thereby increasing motional freedom or fluidity of the lipids. Branched fatty acids, including isovaleric acid, neopentanoic acid, neoheptanoic acid, neonanoic acid, trimethyl hexaonic acid, neodecanoic acid and isostearic acid, are a further class of penetration enhancers. An additional class of penetration enhancers are aliphatic fatty acid esters, such as ethyl oleate, isopropyl n-butyrate, isopropyl n-hexanoate, isopropyl n-decanoate, isopropyl myristate (“IPM”), isopropyl palmitate and octyldodecyl myristate. Aliphatic fatty acid esters enhance penetration by increasing diffusivity in the stratum corneum and/or the partition coefficient. In addition, certain aliphatic fatty acid esters, such as IPM, enhance penetration by directly acting on the stratum corneum and permeating into the liposome bilayers thereby increasing fluidity. Alkyl fatty acid esters, such as ethyl acetate, butyl acetate, methyl acetate, methylvalerate, methylpropionate, diethyl sebacate, ethyl oleate, butyl stearate and methyl laurate, can act as penetration enhancers. Alkyl fatty acid esters enhance penetration in part by increasing the lipid fluidity.

In additional class of penetration enhancers are anionic surfactants, including sodium laurate, sodium lauryl sulfate and sodium octyl sulfate. Anionic surfactants enhance penetration of active agents by altering the barrier function of the stratum corneum and allowing removal of water-soluble agents that normally act as plasticizers. A further class of penetration enhancers are cationic surfactants, such as cetyltrimethylammonium bromide, tetradecyltrimethylammonium, octyltrimethyl ammonium bromide, benzalkonium chloride, octadecyltrimethylammonium chloride, cetylpyridinium chloride, dodecyltrimethylammonium chloride and hexadecyltrimethylammonium chloride. Cationic surfactants enhance penetration by adsorbing at, and interacting with, interfaces of biological membranes, resulting in skin damage. A further class of penetration enhancers are zwitterionic surfactants, such as hexadecyl trimethyl ammoniopropane sulfonate, oleyl betaine, cocamidopropyl hydroxysultaine and cocamidopropyl betaine. Nonionic surfactants, including Polyxamer (231, 182, 184), Polysorbate (20, 60), Brij (30, 93, 96, 99), Span (20, 40, 60, 80, 85), Tween (20, 40, 60, 80), Myrj (45, 51, 52) and Miglyol 840, are yet another class of penetration enhancing agents. Nonionic surfactants enhance penetration in part by emulsifying the sebum and enhancing the thermodynamic activity coefficient of the active.

Further penetration enhancers are bile salts, such as sodium cholate, sodium salts of taurocholic acid, glycolic acids and desoxycholic acids. Lecithin also has been found have penetration enhancing characteristics. An additional class of penetration enhancers are terpenes, which include hydrocarbons, such as d-limonene, alpha-pinene and beta-carene; alcohols, such as, alpha-terpineol, terpinen-4-ol and carvol; ketones, such ascarvone, pulegone, piperitone and menthone; oxides, such as cyclohexene oxide, limonene oxide, alpha-pinene oxide, cyclopentene oxide and 1,8-cineole; and oils such as ylang ylang, anise, chenopodium and eucalyptus. Terpenes enhance penetration in part by disrupting the intercellular lipid bilayer to increase difusivity of the active and opening polar pathways within and across the stratum corneum. Organic acids, such as salicylic acid and salicylates (including their methyl, ethyl and propyl glycol derivates), citric acid and succinic acid, are penetration enhancers. Another class of penetration enhancers are cyclodextrins, including 2-hydroxypropyl-beta-cyclodextrin and 2,6-dimethyl-beta-cyclodextrin. Cyclodextrins enhance the penetration of active agents by forming inclusion complexes with lipophilic actives and increasing their solubility in aqueous solutions.

Additional penetrations enhancers include, but are not limited to: alkyl-2-(N,N-disubstituted amino)-alkanoate ester (NexAct®); 2-(n-nonyl)-1,3-dioxolane (SEPA®); di(lower)alkyl esters of diacids (e.g., diisopropyl adipate); monoglyceride fatty acids (e.g., glyceryl monolaurate); tetrahydrofurfuryl alcohol; 2-(2-ethoxyethoxy)ethanol; alkylaryl ethers of polyethylene oxide; polyethylene oxide monomethyl ethers; polyethylene oxide dimethyl ethers; acetoacetic ester; oleoyl macrogolglyceride; caprylocaproyl macrogolylyceride; polyoxyethylene 6 caprylic triglyceride; polyoxyethylene glyceride; PPG-5 ceteth-20; lauroyl macroglyceride oleic acid. Additional penetration enhancers suitable for use can also be found in U.S. patent application Ser. No. 10/032,163, which is incorporated by reference herein.

The penetration enhancing agent(s) is/are present in an amount sufficient to provide the desired level of drug transport through the stratum corneum and epidermis. Illustratively, one or more pharmaceutically acceptable penetration enhancer is present in a total amount by weight of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9% or about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%.

As a further illustration, one or more pharmaceutically acceptable penetration enhancer is present in a total amount by weight of about 1% to about 95%; about 5% to about 95%; about 10% to about 95%; about 15% to about 95%; about 20% to about 95%; about 25% to about 95%; about 30% to about 95%; about 35% to about 95%; about 40% to about 95%; about 45% to about 95%; about 50% to about 95%; about 55% to about 95%; about 60% to about 95%; about 65% to about 95%; about 70% to about 95%; about 75% to about 95%; about 80% to about 95%; about 85% to about 95%; about 90% to about 95%; about 1% to about 90%; about 5% to about 90%; about 10% to about 90%; about 15% to about 90%; about 20% to about 90%; about 25% to about 90%; about 30% to about 90%; about 35% to about 90%; about 40% to about 90%; about 45% to about 90%; about 50% to about 90%; about 55% to about 90%; about 60% to about 90%; about 65% to about 90%; about 70% to about 90%; about 75% to about 90%; about 80% to about 90%; about 85% to about 90%; about 1% to about 85%; about 5% to about 85%; about 10% to about 85%; about 15% to about 85%; about 20% to about 85%; about 25% to about 85%; about 30% to about 85%; about 35% to about 85%; about 40% to about 85%; about 45% to about 85%; about 50% to about 85%; about 55% to about 85%; about 60% to about 85%; about 65% to about 85%; about 70% to about 85%; about 75% to about 85%; about 80% to about 85%; about 1% to about 80%; about 5% to about 80%; about 10% to about 80%; about 15% to about 80%; about 20% to about 80%; about 25% to about 80%; about 30% to about 80%; about 35% to about 80%; about 40% to about 80%; about 45% to about 80%; about 50% to about 80%; about 55% to about 80%; about 60% to about 80%; about 65% to about 80%; about 70% to about 80%; about 75% to about 80%; about 1% to about 75%; about 5% to about 75%; about 10% to about 75%; about 15% to about 75%; about 20% to about 75%; about 25% to about 75%; about 30% to about 75%; about 35% to about 75%; about 40% to about 75%; about 45% to about 75%; about 50% to about 75%; about 55% to about 75%; about 60% to about 75%; about 65% to about 75%; about 70% to about 75%; about 1% to about 70%; about 5% to about 70%; about 10% to about 70%; about 15% to about 70%; about 20% to about 70%; about 25% to about 70%; about 30% to about 70%; about 35% to about 70%; about 40% to about 70%; about 45% to about 70%; about 50% to about 70%; about 55% to about 70%; about 60% to about 70%; about 65% to about 70%; about 1% to about 65%; about 5% to about 65%; about 10% to about 65%; about 15% to about 65%; about 20% to about 65%; about 25% to about 65%; about 30% to about 65%; about 35% to about 65%; about 40% to about 65%; about 45% to about 65%; about 50% to about 65%; about 55% to about 65%; about 60% to about 65%; about 1% to about 60%; about 5% to about 60%; about 10% to about 60%; about 15% to about 60%; about 20% to about 60%; about 25% to about 60%; about 30% to about 60%; about 35% to about 60%; about 40% to about 60%; about 45% to about 60%; about 50% to about 60%; about 55% to about 60%; about 1% to about 55%; about 5% to about 55%; about 10% to about 55%; about 15% to about 55%; about 20% to about 55%; about 25% to about 55%; about 30% to about 55%; about 35% to about 55%; about 40% to about 55%; about 45% to about 55%; about 50% to about 55%; about 1% to about 50%; about 5% to about 50%; about 10% to about 50%; about 15% to about 50%; about 20% to about 50%; about 25% to about 50%; about 30% to about 50%; about 35% to about 50%; about 40% to about 50%; about 45% to about 50%; about 1% to about 45%; about 5% to about 45%; about 10% to about 45%; about 15% to about 45%; about 20% to about 45%; about 25% to about 45%; about 30% to about 45%; about 35% to about 45%; about 40% to about 45%; about 1% to about 40%; about 5% to about 40%; about 10% to about 40%; about 15% to about 40%; about 20% to about 40%; about 25% to about 40%; about 30% to about 40%; about 35% to about 40%; about 1% to about 35%; about 5% to about 35%; about 10% to about 35%; about 15% to about 35%; about 20% to about 35%; about 25% to about 35%; about 30% to about 35%; about 1% to about 30%; about 5% to about 30%; about 10% to about 30%; about 15% to about 30%; about 20% to about 30%; about 25% to about 30%; about 1% to about 25%; about 5% to about 25%; about 10% to about 25%; about 15% to about 25%; about 20% to about 25%; about 1% to about 20%; about 5% to about 20%; about 10% to about 20%; about 15% to about 20%; about 1% to about 15%; about 5% to about 15%; or about 10% to about 15%; about 1% to about 10%; about 2% to about 10%; about 3% to about 10%; about 4% to about 10%; about 5% to about 10%; about 6% to about 10%; about 7% to about 10%; about 8% to about 10%; about 9% to about 10%; about 1% to about 9%; about 2% to about 9%; about 3% to about 9%; about 4% to about 9%; about 5% to about 9%; about 6% to about 9%; about 7% to about 9%; about 8% to about 9%; about 1% to about 8%; about 2% to about 8%; about 3% to about 8%; about 4% to about 8%; about 5% to about 8%; about 6% to about 8%; about 7% to about 8%; about 1% to about 7%; about 2% to about 7%; about 3% to about 7%; about 4% to about 7%; about 5% to about 7%; about 6% to about 7%; about 1% to about 6%; about 2% to about 6%; about 3% to about 6%; about 4% to about 6%; about 5% to about 6%; about 1% to about 5%; about 2% to about 5%; about 3% to about 5%; about 4% to about 5%; about 1% to about 4%; about 2% to about 4%; about 3% to about 4%; about 1% to about 3%; about 2% to about 3%; or about 1% to about 2%.

In one embodiment, the cannabidiol can be combined with a thickening or gelling agent suitable for use in the compositions and methods described herein to increase the viscosity of the composition. Non-limiting examples of thickening agents (aka gelling agents) which may be used to create the composition or be present in the composition herein include neutralized anionic polymers or neutralized carbomers such as polyacrylic acid (CARBOPOL® by Noveon, Inc., Cleveland, Ohio) (see information at http://www.nuven.com, incorporated by reference herein), carboxypolymethylene, carboxymethylcellulose and the like, including derivatives of Carbopol® polymers, such as Carbopol® Ultrez 10, Carbopol® 940, Carbopol® 941, Carbopol® 954, Carbopol® 980, Carbopol® 981, Carbopol® ETD 2001, Carbopol® EZ-2 and Carbopol® EZ-3. As used herein, a “neutralized carbomer” is a synthetic, high molecular weight polymer, composed primarily of a neutralized polyacrylic acid. Further, when a base is added to neutralize a carbomer solution, the viscosity of the solution increases. Also suitable are other known polymeric thickening agents such as Pemulen® polymeric emulsifiers, Noveon® polycarbophils, and Klucel®. Additional thickening agents, enhancers and adjuvants may generally be found in Remington's The Science and Practice of Pharmacy as well as the Handbook of Pharmaceutical Excipients, Arthur H. Kibbe ed. 2000. Thickening agents or gelling agents are present in an amount sufficient to provide the desired rheological properties of the composition, which include having a sufficient viscosity for forming a gel or gel-like composition that can be applied to the skin of a mammal.

Illustratively, one or more pharmaceutically acceptable thickening agent or gelling agent is present in a total amount by weight of about 0.1%, about 0.25%, about 0.5%, about 0.75%, about 1%, about 1.25%, about 1.5%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, about 5.0%, about 5.25%, about 5.5%, about 5.75%, about 6.0%, about 6.25%, about 6.5%, about 6.75%, about 7.0%, about 7.25%, about 7.5%, about 7.75%, about 8.0%, about 8.25%, about 8.5%, about 8.75%, about 9.0%, about 9.25%, about 9.5%, about 9.75%, about 10%, about 11%, about 11.5%, about 12%, about 12.5%, about 13%, about 13.5%, about 14%, about 14.5% or about 15%. As a further illustration, one or more pharmaceutically acceptable thickening or gelling agent is present in a total amount by weight of about 0.1% to about 15.0%; about 0.5% to about 5.0%; or about 1.0% to about 3.0%.

In one embodiment a neutralizing agent is optionally used to assist in forming a gel or gel-like composition. Suitable neutralizing agents include sodium hydroxide (e.g., as an aqueous mixture), potassium hydroxide (e.g., as an aqueous mixture), ammonium hydroxide (e.g., as an aqueous mixture), triethanolamine, tromethamine (2-amino 2-hydroxymethyl-1,3 propanediol), aminomethyl propanol (AMP), tetrahydroxypropyl ethylene diamine, diisopropanolamine, Ethomeen C-25 (Armac Industrial Division), Di-2 (ethylhexyl) amine (BASF-Wyandotte Corp., Intermediate Chemicals Division), triamylamine, Jeffamine D-1000 (Jefferson Chemical Co.), b-Dimethylaminopropionitrite (American Cyanamid Co.), Armeen CD (Armac Industrial Division), Alamine 7D (Henkel Corporation), dodecylamine and morpholine. The neutralizing agent is present in an amount sufficient to increase viscosity and form a gel or gel-like composition which is suitable for contact with the skin of a mammal. Illustratively, one or more pharmaceutically acceptable neutralizing agent is present in a total amount by weight of about 0.001%, about 0.0015%, about 0.01%, about 0.015%, about 0.1%, about 0.2%, about 0.3%, about 0.4%, about 0.5%, about 0.6%, about 0.7%, about 0.8%, about 0.9%, about 1.0%, 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%. As a further illustration, one or more pharmaceutically acceptable neutralizing agent is present in a total amount by weight of about 0.1% to about 7.0% or about 1.0% to about 5.0%.

In one embodiment, a solution of sodium hydroxide is used, such as, e.g., 0.01 N, 0.02 N, 0.025 N, 0.05 N, 0.075 N, 0.1 N sodium hydroxide solution, 0.2 N sodium hydroxide solution, 0.5 N sodium hydroxide solution, 1.0 N sodium hydroxide solution, 1.5 N sodium hydroxide solution, 2.0 N sodium hydroxide solution, 10.0 N sodium hydroxide solution, or any other suitable solution for providing a sufficient amount of the aqueous sodium hydroxide to form the desired gel or gel-like composition. In one embodiment, the composition results from combining a gelling agent with a neutralizing agent such as about 1% to about 10% (wt/wt) 0.025 N sodium hydroxide, while in another embodiment about 0.1% to about 1% (wt/wt) 0.25 N sodium hydroxide is used. Of course, other suitable neutralizing agents can be used as can other concentrations and amounts of aqueous sodium hydroxide so long as there is a sufficient amount of Off ions to assist in the formation of a gel or gel-like composition.

Compositions described herein optionally comprise one or more pharmaceutically acceptable wetting agents as excipients. Non-limiting examples of surfactants that can be used as wetting agents in compositions of the disclosure include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefosse), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters (e.g., sorbitan monolaurate, sorbitan monooleate, sorbitan monopalmitate and sorbitan monostearate), tyloxapol, and mixtures thereof. Such wetting agents, if present, constitute in total about 0.25% to about 15%, about 0.4% to about 10%, or about 0.5% to about 5%, of the total weight of the composition. Illustratively, one or more pharmaceutically acceptable wetting agents are present in a total amount by weight of about 0.25%, about 0.5%, about 0.75%, about 1%, about 1.25%, about 1.5%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, about 5.0%, about 5.25%, about 5.5%, about 5.75%, about 6.0%, about 6.25%, about 6.5%, about 6.75%, about 7.0%, about 7.25%, about 7.5%, about 7.75%, about 8.0%, about 8.25%, about 8.5%, about 8.75%, about 9.0%, about 9.25%, about 9.5%, about 9.75% or about 10%.

As used herein, a “solubility agent” is any excipient which is added to a pharmaceutical composition to increase the solubility of a solute.

Compositions described herein optionally comprise one or more pharmaceutically acceptable lubricant, including an anti-adherent and/or a glidant. Suitable lubricants include, either individually or in combination, glyceryl behapate (e.g., Compritol™ 888); stearic acid and salts thereof, including magnesium (magnesium stearate), calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; polyethylene glycol (“PEG”) (e.g., Carbowax™ 4000 and Carbowax™ 6000); sodium oleate; sodium lauryl sulfate; and magnesium lauryl sulfate. Such lubricants, if present, constitute about 0.1% to about 10%, about 0.2% to about 8%, or about 0.25% to about 5%, of the total weight of the composition. Illustratively, one or more pharmaceutically acceptable lubricant is present in a total amount by weight of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9% or about 10%.

In another embodiment, the compositions described herein optionally comprise an emollient. Illustrative emollients include mineral oil, mixtures of mineral oil and lanolin alcohols, cetyl alcohol, cetostearyl alcohol, petrolatum, petrolatum and lanolin alcohols, cetyl esters wax, cholesterol, glycerin, glyceryl monostearate, isopropyl myristate, isopropyl palmitate, lecithin, allyl caproate, althea officinalis extract, arachidyl alcohol, argobase EUC, butylene glycol, dicaprylate/dicaprate, acacia, allantoin, carrageenan, cetyl dimethicone, cyclomethicone, diethyl succinate, dihydroabietyl behenate, dioctyl adipate, ethyl laurate, ethyl palmitate, ethyl stearate, isoamyl laurate, octanoate, PEG-75, lanolin, sorbitan laurate, walnut oil, wheat germ oil, super refined almond, super refined sesame, super refined soyabean, octyl palmitate, caprylic/capric triglyceride and glyceryl cocoate. An emollient, if present, is present in the compositions described herein in an amount by weight of the composition of about 1% to about 30%, about 3% to about 25%, or about 5% to about 15%. Illustratively, one or more emollients are present in a total amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%, by weight.

In one embodiment, the compositions described herein comprise an antioxidant. Illustrative antioxidants include citric acid, butylated hydroxytoluene (BHT), ascorbic acid, glutathione, retinol, α-tocopherol, β-carotene, α-carotene, ubiquinone, butylated hydroxyanisole, ethylenediaminetetraacetic acid, selenium, zinc, lignan, uric acid, lipoic acid, and N-acetylcysteine. An antioxidant, if present, is present in the compositions described herein in the amount of about less than 1% by weight. Illustratively, one or more antioxidants are present in the total amount of about 0.025%, about 0.05%, about 0.075%, about 0.1%, about 0.125%, about 0.15%, about 0.175%, about 0.2%, about 0.225%, about 0.25%, about 0.275%, about 0.3%, 0.325%, about 0.35%, about 0.375%, about 0.4%, about 0.425%, about 0.45%, about 0.475%, about 0.5%, about 0.525%, about 0.55%, about 0.575%, about 0.6%, about 0.625%, about 0.65%, about 0.675%, about 0.7%, about 0.725%, about 0.75%, about 0.775%, about 0.8%, about 0.825%, about 0.85%, about 0.875%, about 0.9%, about 0.925%, about 0.95%, about 0.975%, or about 1.0%, by weight. As a further illustration one or more antioxidants are present in the total amount by weight of about 0.01% to about 1.0%; about 0.05% to about 0.5% or about 0.05% to about 0.2%.

In one embodiment, the compositions described herein comprise an antimicrobial preservative. Illustrative anti-microbial preservatives include acids, including but not limited to benzoic acid, phenolic acid, sorbic acids, alcohols, benzethonium chloride, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, propylparaben, sodium propionate, or thimerosal. The anti-microbial preservative, if present, is present in an amount by weight of the composition of about 0.1% to about 5%, about 0.2% to about 3%, or about 0.3% to about 2%, for example about 0.2%, about 0.4%, about 0.6%, about 0.8%, about 1%, about 1.2%, about 1.4%, about 1.6%, about 1.8%, about 2%, about 2.2%, about 2.4%, about 2.6%, about 2.8%, about 3.0%, about 3.2%, about 3.4%, about 3.6%, about 3.8%, about 4%, about 4.2%, about 4.4%, about 4.6%, about 4.8%, or about 5%.

Compositions described herein optionally comprise one or more emulsifying agents. The term “emulsifying agent” refers to an agent capable of lowering surface tension between a non-polar and polar phase and includes compounds defined elsewhere as “self emulsifying” agents. Suitable emulsifying agents can come from any class of pharmaceutically acceptable emulsifying agents including carbohydrates, proteins, high molecular weight alcohols, wetting agents, waxes and finely divided solids. The optional emulsifying agent, if present, is present in a composition in a total amount of about 1% to about 25%, about 1% to about 20%, or about 1% to about 15% by weight of the composition. Illustratively, one or more emulsifying agents are present in a total amount by weight of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%.

In another embodiment, the composition optionally comprises a water miscible solvent, such as propylene glycol. A suitable water miscible solvent refers to any solvent that is acceptable for use in a pharmaceutical composition and is miscible with water. If present, the water miscible solvent is present in a composition in a total amount of about 1% to about 95%, about 2% to about 75%, about 3% to about 50%, about 4% to about 40%, or about 5% to about 25% by weight of the composition. In a further embodiment, the water miscible solvent is present in a composition in an amount of about 1% to about 99%, by weight of the composition, for example about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 99%.

Compositions described herein may optionally comprise one or more alcohols. In a further embodiment, the alcohol is a lower alcohol. As used herein, the term “lower alcohol,” alone or in combination, means a straight-chain or branched-chain alcohol moiety containing one to about six carbon atoms. In one embodiment, the lower alcohol contains one to about four carbon atoms, and in another embodiment the lower alcohol contains two or three carbon atoms. Examples of such alcohol moieties include methanol, ethanol, ethanol USP (i.e., 95% v/v), n-propanol, isopropanol, n-butanol, isobutanol, sec-butanol, and tert-butanol. As used herein, the term “ethanol” refers to C₂H₅OH. It may be used as dehydrated alcohol USP, alcohol USP or in any common form including in combination with various amounts of water. If present, the alcohol is present in an amount sufficient to form a composition which is suitable for contact with a mammal. Illustratively, one or more pharmaceutically acceptable alcohol is present in a total amount by weight of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, or about 98%. As a further illustration, one or more pharmaceutically acceptable alcohol is present in a total amount by weight of about 1% to about 98%; about 10% to about 95%; about 25% to about 75%; about 35% to about 70%; or about 40% to about 50%.

In a further embodiment water is separately added to the composition. The amount of water separately added to a formulation is exclusive of the amount of water independently present in the formulation from any other component (e.g., alcohol, neutralizing agent). Water is present in an amount sufficient to form a composition which is suitable for administration to a mammal. Illustratively, water can be separately added by weight in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90% about 91%, about 92%, about 93%, about 94%, about 95%, about 96%, about 97%, or about 98%. As a further illustration, water can be separately added by weight in an amount of about 1% to about 98%; about 10% to about 70%; about 10% to about 40%; about 10% to about 30%; about 20% to about 30%; or about 25% to about 30%.

In a further embodiment, water is separately added to the composition in a quantity or amount sufficient to achieve the desired weight of the composition. In an additional embodiment, water is separately added in a quantity sufficient to obtain 100% weight of the composition.

Pharmaceutical Dosage Forms

The compositions described herein are used in a “pharmacologically effective amount.” A “pharmacologically effective amount” is the amount of the active pharmaceutical agent in the composition which is sufficient to deliver a therapeutic amount of the active agent during the dosing interval in which the composition is administered.

In one embodiment, the amount of the pharmaceutical composition administered to deliver a therapeutically effective amount of the cannabinoid is about 0.1 g, about 0.2 g, about 0.3 g, about 0.4 g, about 0.5 g, about 0.6 g, about 0.7 g, about 0.8 g, about 0.9 g, about 1 g, about 1.1 g, about 1.2 g, about 1.3 g, about 1.4 g, about 1.5 g, about 1.6 g, about 1.7 g, about 1.8 g, about 1.9 g, about 2 g, about 2.1 g, about 2.2 g, about 2.3 g, about 2.4 g, about 2.5 g, about 2.6 g, about 2.7 g, about 2.8 g, about 2.9 g, about 3 g, about 3.1 g, about 3.2 g, about 3.3 g, about 3.4 g, about 3.5 g, about 3.6 g, about 3.7 g, about 3.8 g, about 3.9 g, about 4 g, about 4.1 g, about 4.2 g, about 4.3 g, about 4.4 g, about 4.5 g, about 4.6 g, about 4.7 g, about 4.8 g, about 4.9 g, about 5 g, about 5.1 g, about 5.2 g, about 5.3 g, about 5.4 g, about 5.5 g, about 5.6 g, about 5.7 g, about 5.8 g, about 5.9 g, about 6 g, about 6.1 g, about 6.2 g, about 6.3 g, about 6.4 g, about 6.5 g, about 6.6 g, about 6.7 g, about 6.8 g, about 6.9 g, about 7 g, about 7.1 g, about 7.2 g, about 7.3 g, about 7.4 g, about 7.5 g, about 7.6 g, about 7.7 g, about 7.8 g, about 7.9 g, about 8 g, about 8.1 g, about 8.2 g, about 8.3 g, about 8.4 g, about 8.5 g, about 8.6 g, about 8.7 g, about 8.8 g, about 8.9 g, about 9 g, about 9.1 g, about 9.2 g, about 9.3 g, about 9.4 g, about 9.5 g, about 9.6 g, about 9.7 g, about 9.8 g, about 9.9 g or about 10 g.

Illustratively, the amount of the pharmaceutical composition administered to deliver a therapeutically effective amount of the cannabinoid is about 1 g to about 10 g, about 1 g to about 6 g, about 1 g to about 2 g, or about 2 g to about 4 g.

In one embodiment, the formulation is a gel, gel-like composition, an ointment, a cream or a patch and comprises cannabidiol, optionally one or more penetration enhancing agents, such as transcutol, isopropyl myristate or propylene glycol; a thickening agent, such as neutralized carbomer; a lower alcohol, such as ethanol or isopropanol; and water. In another embodiment, the formulation is a gel, gel-like composition, an ointment, a cream or a patch, further comprised of an aqueous solution of sodium hydroxide or triethanolamine or an aqueous solution of potassium hydroxide, or a combination thereof, in an amount sufficient, as is known in the art, to assist the gelling agent in forming a gel or gel-like composition.

In another embodiment, the formulation contains an anionic polymer thickening agent precursor such as a carbomer to be combined with a neutralizer in an amount sufficient to form a gel or gel-like composition in the course of forming the composition.

In another embodiment, the formulation contains an anionic polymer thickening agent precursor such as a carbomer which has been combined with a neutralizer in an amount sufficient to form a gel or gel-like composition with a viscosity greater than 1000 cps as measured by a Brookfield RV DVII+ Viscometer with spindle CPE-52, torque greater than 10%, and the temperature maintained at 25° C.

In yet a further embodiment, the formulation contains an anionic polymer thickening agent precursor such as a carbomer which has been combined with a neutralizer selected from the group consisting of sodium hydroxide, ammonium hydroxide, potassium hydroxide, arginine, aminomethyl propanol, tetrahydroxypropyl ethylenediamine, triethanolamine (“TEA”), tromethamine, PEG-15 cocamine, diisopropanolamine, and triisopropanolamine, or combinations thereof in an amount sufficient to neutralize the anionic polymer thickening agent precursor to form a gel or gel-like composition in the course of forming the composition. Suitable neutralizing agents and their use with selected anionic polymer thickening agent precursors are disclosed in “Neutralizing Carbopol® and Pemulen® Polymers in Aqueous and Hydroalcoholic Systems,” Commercial Brochure TDS-237 (October 1998) by Noveon Inc. of Cleveland, Ohio, incorporated by reference herein.

In yet a further embodiment, the formulation contains an anionic polymer thickening agent precursor such as a carbomer which has been combined with a neutralizer which is an aqueous solution of sodium hydroxide such as 0.01 N, 0.02 N, 0.025 N, 0.05 N, 0.075 N, 0.1 N sodium hydroxide, or 1.5 N sodium hydroxide, or 2.0 N sodium hydroxide or any other convenient strength aqueous solution in an amount sufficient to adequately neutralize the polyacrylic acid and form a gel or gel-like composition. In one embodiment, the composition was prepared using from about 1.0% to about 10.0% 0.025N sodium hydroxide. Accordingly, embodiments employing any percentage from about 1.0% to about 10.0% 0.025 N NaOH may be used, such as, e.g., 1.0%, 2.0%, 3.0%, 4.0%, 5.0%, 6.0%, 7.0%, 8.0%, 9.0% or 10% 0.025 N NaOH.

In an embodiment, the viscosity of a composition described herein is about 1,000 cps to about 100,000 cps. Accordingly, the viscosity of the compositions described and disclosed herein may be any amount from about 1,000 cps to about 100,000 cps, such as, e.g., about 1,000, about 2,000, about 3,000, about 4,000, about 5,000, about 6,000, about 7,000, about 8,000, about 9,000, about 10,000, about 11,000, about 12,000, about 13,000, about 14,000, about 15,000, about 16,000, about 17,000, about 18,000, about 19,000, about 20,000, about 21,000, about 22,000, about 23,000, about 24,000, about 25,000, about 26,000, about 27,000, about 28,000, about 29,000, about 30,000, about 31,000, about 32,000, about 33,000, about 34,000, about 35,000, about 36,000, about 37,000, about 38,000, about 39,000, about 40,000, about 41,000, about 42,000, about 43,000, about 44,000, about 45,000, about 46,000, about 47,000, about 48,000, about 49,000, about 50,000, about 51,000, about 52,000, about 53,000, about 54,000, about 55,000, about 56,000, about 57,000, about 58,000, about 59,000, about 60,000, about 61,000, about 62,000, about 63,000, about 64,000, about 65,000, about 66,000, about 67,000, about 68,000, about 69,000, about 70,000, about 71,000, about 72,000, about 73,000, about 74,000, about 75,000, about 76,000, about 77,000, about 78,000, about 79,000, about 80,000, about 81,000, about 82,000, about 83,000, about 84,000, about 85,000, about 86,000, about 87,000, about 88,000, about 89,000, about 90,000, about 91,000, about 92,000, about 93,000, about 94,000, about 95,000, about 96,000, about 97,000, about 98,000, about 99,000, about 100,000 cps.

In one embodiment, the pH of the pharmaceutical composition is suitable for administration to a mammal. In a further embodiment, the pH of the pharmaceutical composition is suitable for administration to the skin of a mammal. In additional embodiments, the pH of the pharmaceutical composition is suitable for buccal, sublingual, injection, rectal, vaginal, ocular, nasal or oral administration to a mammal. In one embodiment, the pH of the pharmaceutical composition is about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 9.1, about 9.2, about 9.3, about 9.4, about 9.5, about 9.6, about 9.7, about 9.8, about 9.9 or about 10. Illustratively, the pH of the pharmaceutical composition may be from about 3 to about 10, about 4 to about 8, about 4.5 to about 6.5, or about 5 to about 6.

In one embodiment, a single dosage unit of any formulation comprises a therapeutically effective amount or a therapeutically and/or prophylactically effective amount of cannabidiol.

In one embodiment, compositions described herein are suitable for transdermal administration. In another embodiment, transdermally administrable compositions are adapted for administration in and/or around the abdomen, back, chest, legs, arms, scalp or other suitable skin surface and may include formulations in which the cannabidiol is administered in patches, ointments, creams, suspensions, lotions, pastes, gels, sprays, foams or oils.

In another embodiment, compositions described herein which are transdermally administrable include formulations in which the cannabidiol is placed in a glycol, gel or gel-like formulation.

In one embodiment, compositions described herein are suitable for topical administration. In another embodiment, topical administrable compositions are adapted for administration in and/or around the abdomen, back, chest, legs, arms, scalp or other suitable skin surface and may include formulations in which the cannabidiol is administered in patches, ointments, creams, suspensions, lotions, pastes, gels, sprays, foams or oils.

In one embodiment described herein employs a packet having a polyethylene liner compatible with the components of a cannabidol gel or gel-like composition, as described below. The packet may hold a unit dose or multiple dose.

In another embodiment, the methods and compositions employ a composition that is dispensed from a rigid multi-dose container (for example, with a hand pump) having a larger foil packet, for example, of the composition inside the container. Such larger packets can also comprise a polyethylene liner as above. In one embodiment, the multi-dose container comprises an airless pump that comprises a polyethylene lined foil pouch within a canister with a hand pump inserted. In one embodiment, the pump is primed before use, such as, e.g., by fully depressing the pump three times and discarding the gel. In one embodiment, the pump contains enough product to allow for priming and a set number of precise doses. Each pump depression can deliver any amount of cannabidiol suitable for delivering the desired dose. The pouch size, amount dispensed and the delivery volume per depression are not limited to these embodiments and may be changed or adjusted to meet the needs of the patient population.

In one embodiment, the pharmaceutical composition is a gel or gel-like composition, comprising, by weight: 66% EtOH, 21% H₂0, 6% transcutol, 1% CBD, 0.5% IPM, 1.0% gelling agent (e.g., a carbomer, such as Carbopol 980) and 4.5% NaOH (0.1%).

In one embodiment, the pharmaceutical composition is a gel or gel-like composition, comprising, by weight: 63.27% EtOH, 19.73% H₂0, 6% transcutol, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 59.8% EtOH, 18.2% H₂0, 6% transcutol, 10% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 66% EtOH, 20.5% H₂0, 2.5% transcutol, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 63.5% EtOH, 20.5% H₂00, 5% transcutol, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 61% EtOH, 20.5% H₂0, 7.5% transcutol, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 43.5% EtOH, 20.5% H₂0, 5% transcutol, 20% PEG 550, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 56% EtOH, 20.5% H₂0, 7.5% transcutol, 5% PG, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 51% EtOH, 20.5% H₂0, 7.5% transcutol, 10% PG, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 46% EtOH, 20.5% H₂0, 7.5% transcutol, 15% PG, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 53.5% EtOH, 20.5% H₂0, 15% PG, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 49.75% EtOH, 20.5% H₂0, 3.75% transcutol, 15% PG, 5% CBD, 0.5% IPM, 1.0% gelling agent (e.g., a carbomer, such as Carbopol 980), and 4.5% NaOH (0.1%).

In one embodiment, the pharmaceutical composition is a gel or gel-like composition, comprising, by weight: 54.5% EtOH, 15.0% H₂0, 7.5% transcutol, 15% PG, 5% CBD, 0.5% IPM, 0.1% butylated hydroxytoluene, 1.0% gelling agent (e.g., a carbomer, such as Carbopol 980), and 1.4% NaOH (0.1%).

In one embodiment, the pharmaceutical composition is a gel or gel-like composition, comprising, by weight: 54.5% EtOH, 13.9% H₂0, 7.5% transcutol, 15% PG, 5% CBD, 0.5% IPM, 0.1% butylated hydroxytoluene, 0.1% citric acid, 2.0% gelling agent (e.g. Klucel NF), and 1.4% NaOH (0.1%).

In one embodiment, the pharmaceutical composition is a gel or gel-like composition, comprising, by weight: 54.5% EtOH, 15.71% H₂0, 7.5% transcutol, 15% PG, 5% CBD, 0.5% IPM, 0.1% butylated hydroxytoluene, 0.05% citric acid, 1.5% gelling agent (e.g., a carbomer, such as Carbopol 980), and 0.14% triethanolamine.

In one embodiment, the pharmaceutical composition is a gel or gel-like composition, comprising, by weight: 45% EtOH, 27.96% H₂0, 7.5% transcutol, 15% PG, 2.5% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 43.55% EtOH, 27% H₂0, 7.5% transcutol, 15% PG, 5% CBD, 0.5% IPM, 0.1% butylated hydroxytoluene, 0.05% citric acid, 1.25% gelling agent (e.g., a carbomer, such as Carbopol 980), and 0.14% NaOH (1.0%).

In one embodiment, the pharmaceutical composition is a gel or gel-like composition, comprising, by weight: 45.93% EtOH, 28.53% H₂0, 7.5% transcutol, 15% PG, 1.0% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 44.07% EtOH, 27.39% H₂0, 7.5% transcutol, 15% PG, 4.0% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 45% EtOH, 26.21% H₂0, 7.5% transcutol, 15% PG, 4.0% CBD, 0.5% IPM, 0.1% butylated hydroxytoluene, 0.05% citric acid, 1.5% gelling agent (e.g., a carbomer, such as Carbopol 980), and 0.14% triethanolamine.

In one embodiment, the pharmaceutical composition is a gel or gel-like composition, comprising, by weight: 40% EtOH, 26.21% H₂0, 7.5% transcutol, 20% PG, 4.0% CBD, 0.5% IPM, 0.1% butylated hydroxytoluene, 0.05% citric acid, 1.5% gelling agent (e.g., a carbomer, such as Carbopol 980), and 0.14% triethanolamine.

In one embodiment, the pharmaceutical composition is a gel or gel-like composition, comprising, by weight: 40% EtOH, 26.46% H₂0, 7.5% transcutol, 20% PG, 4.0% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 42% EtOH, 26.46% H₂0, 3.5% transcutol, 22% PG, 4.0% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 40% EtOH, 26.46% H₂0, 10% transcutol, 17.5% PG, 4.0% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 45% EtOH, 27.96% H₂0, 3.5% transcutol, 19% PG, 2.5% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 45% EtOH, 26.46% H₂0, 3.5% transcutol, 19% PG, 4.0% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 51% EtOH, 30.96% H₂0, 3.5% transcutol, 10% PG, 2.5% CBD, 0.5% IPM, 0.1% butylated hydroxytoluene, 0.05% citric acid, 1.25% gelling agent (e.g., a carbomer, such as Carbopol 980), and 0.14% triethanolamine.

In one embodiment, the pharmaceutical composition is a gel or gel-like composition, comprising, by weight: 69.88% EtOH, 14.24% H₂0, 10% CBD, 0.47% IPM, 0.86% gelling agent (e.g., a carbomer, such as Carbopol 980), and 4.55% NaOH.

In one embodiment, the pharmaceutical composition is a gel or gel-like composition, comprising, by weight: 72.5% EtOH, 20.4% H₂0, 1% CBD, 0.5% IPM, 0.9% gelling agent (e.g., a carbomer, such as Carbopol 980), and 4.7% NaOH (0.1 N).

The cannabinoid may also be applied with another pain relieving analgesic composition, e.g., an opiate, NSAID or COX-2 specific inhibitor, wherein the concentration of the other analgesic composition is sufficient to further alleviate the symptoms associated with osteoarthritis. The analgesic may be combined with the cannabinoid and the gel and applied to the skin of a subject in need thereof.

The cannabinoid or cannabinoid-containing composition is applied to the subject's skin in an amount and for a time sufficient to alleviate the symptoms, e.g., inflammation, pain and/or discomfort, that are associated with an injury e.g. a strain, sprain, contusion, or or disease condition, e.g., arthritis, particularly osteoarthritis. The cannabinoid or cannabinoid-containing composition may be applied in a single dose or multiple doses for one or more days. The cannabinoid-containing composition may be applied to the skin of a mammal for a time and in an amount to deliver about 5-250 mg/day, about 5 mg-100 mg/day, about 10 mg-50 mg, about 25-40 mg/day or about 36 mg/day of the cannabinoid to the mammal. Preferably the cannabinoid-containing composition is applied once or twice per day to alleviate the subject's symptoms.

Although the cannabinoid-containing composition may be applied to skin in the area affected by the injury or disease condition, e.g., an osteoarthritic joint, it may also be applied to other areas of the body distant or not immediately proximate to the area affected by the injury or disease condition, e.g., the subject's back, abdomen, chest, upper arms, thighs etc., and still deliver the desired amounts of cannabinoid to the subject. The cannabinoid-containing composition may be applied to the subject's skin until it is completely or almost completely, absorbed into the skin.

Another aspect of the invention provides a method for increasing the concentration of cannabinoids or cannabinoid metabolites in a subject, comprising contacting the subject's skin with a cannabinoid selected from the group consisting of cannabinol, cannabidiol, nabilone, levonantradol, (−)-HU-210, (+)-HU-210, 11-hydroxy-Δ⁹-THC, Δ⁸-THC-11-oic acid, CP 55,940, R(+)-WIN 55, 212-2 and Δ⁹-THC. The method may also comprise the step of contacting the subject's skin with a composition comprising the cannabinoid, an alcohol having one to six carbons, water and a penetration enhancer (e.g., isopropyl myristate, propylene glycol monolaurate, diethylene glycol monoethyl ether, an oleoyl macrogolglyceride, a caprylocaproyl macrogolglyceride, and an oleyl alcohol, and oleic acid). The cannabinoid, with or without the penetration enhancer, is preferably a component of a pharmaceutically acceptable gel or gel-like vehicle. The gel or gel-like composition may be applied to the subject's skin until it is completely or almost completely, absorbed into the skin.

“Alleviate” as used herein, is meant to include complete elimination as well as any clinically or quantitatively measurable reduction in the subject's symptoms and/or discomfort.

“Cannabinoid,” as used herein, is meant to include compounds which interact with the cannabinoid receptor and various cannabinoid mimetics, such as certain tetrahydropyran analogs (e.g., Δ⁹-tetrahydrocannabinol, Δ⁸-tetrahydrocannabinol, 6,6,9-trimethyl-3-pentyl-6H-dibenzo [b,d]pyran-1-ol, 3-(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-Δ⁶-tetrahydrocannabinol-1,1-dimethylhept-yl, (+)-(3S,4S)-7-hydroxy-Δ⁶-tetrahydrocannabinol-1,1-dimethylh-eptyl, 11-hydroxy-Δ⁹-tetrahydrocannabinol, and Δ⁸-tetrahydrocannabinol-11-oic acid)); certain piperidine analogs (e.g., (−)-(6S,6aR,9R,10aR)-5,6,6a,7,8,9,10,10a-octahydro-6-methyl-1-3-[(R)-1-methyl-4-phenylbutoxy]-1,9-phenanthridinediol 1-acetate)), certain aminoalkylindole analogs (e.g., (R)-(+)-[2,3-dihydro-5-methyl-3-(−4-morpholinylmethyl)-pyrrolo[1,2,3-de]-1,4-benzoxazin-6-yl]-1-naphthalenylmethanone), certain open pyran ring analogs (e.g., 2-[3-methyl-6-(1-methylethenyl)-2-cyclohexen-1-yl]-5-pentyl-1,3-benzenedi-ol and 4-(1,1-dimethylheptyl)-2,3′-dihydroxy-6′ alpha-(3-hydroxypropyl)-1′,-2′,3′,4′,5′,6′-hexahydrobiphen-yl), as well as their pharmaceutically acceptable salts, solvates, metabolites (e.g., cutaneous metabolites), and metabolic precursors. Further examples of “cannabinoids” include those compounds described in the references cited below.

“Δ⁹-THC,” as used herein, is meant to refer to Δ⁹-tetrahydrocannabinol as well as to its pharmaceutically acceptable salts, solvates, metabolites (e.g., cutaneous metabolites), and metabolic precursors. Δ⁹-tetrahydrocannabinol is marketed under the generic name “dronabinol.”

“Cannabinol,” as used herein, is meant to refer to 6,6,9-trimethyl-3-pentyl-6H-dibenzo[b,d]pyran-1-ol as well as to pharmaceutically acceptable salts, solvates, metabolites (e.g., cutaneous metabolites), and metabolic precursors of 6,6,9-trimethyl-3-pentyl-6H-dib-enzo[b,d]pyran-1-ol. The synthesis of 6,6,9-trimethyl-3-pentyl-6H-dibenzo[-b,d]pyran-1-ol is described in, for example, Novak et al., Tetrahedron Letters, 23:253 (1982), which is hereby incorporated by reference.

“Cannabidiol,” as used herein, is meant to refer to 2-[3-methyl-6-(1-methylethenyl)-2-cyclohexen-1-yl]-5-pentyl-1,3-benzenedi-ol as well as to pharmaceutically acceptable salts, solvates, metabolites (e.g., cutaneous metabolites), and metabolic precursors of 2-[3-methyl-6-(1-methylethenyl)-2-cyclohexen-1-yl]-5-pentyl-1,3-benzenedi-ol. The synthesis of 2-[3-methyl-6-(1-methylethenyl)-2-cyclohexen-1-yl]-5-pentyl-1,3-benzenediol is described, for example, in Petilka et al., Helv. Chim. Acta, 52:1102 (1969) and in Mechoulam et al., J. Am. Chem. Soc., 87:3273 (1965), which are hereby incorporated by reference.

“Nabilone,” as used herein, is meant to refer to 3-(1,1-dimethylheptyl)-6,6a,7,8,10,10a-hexahydro-1-hydroxy-6,6-dimethyl-9-H-dibenzo [b,d]pyran-9-one as well as to pharmaceutically acceptable salts, solvates, metabolites (e.g., cutaneous metabolites), and metabolic precursors of 3-(1,1-dimethylheptyl)-6,6a,7,8,10,10a-hexahydro-1-hydroxy-1-6,6-dimethyl-9H-dibenzo[b,d]pyran-9-one. 3-(1,1-dimethylheptyl)-6,6a,7,8,-10,10a-hexahydro-1-hydroxy-6,6-dimethyl-9H-dibenzo[b,d]pyran-9-one is approved for use in the United Kingdom for treating nausea and vomiting associated with chemotherapy, and its preparation is described, for example, in U.S. Pat. No. 3,968,125 to Archer, which is hereby incorporated by reference.

“Levonantradol,” as used herein, is meant to refer to (−)-(6S,6aR,9R,10aR)-5,6,6a,7,8,9,10,10a-octahydro-6-methyl-3-[(R)-1-meth-yl-4-phenylbutoxy]-1,9-phenan-thridinediol 1-acetate, as well as to pharmaceutically acceptable salts, solvates, metabolites (e.g., cutaneous metabolites), and metabolic precursors of (−)-(6S,6aR,9R, OaR)-5,6,6a,7,8,9,10,10a-octahydro-6-methyl-3-[(R)-1-methyl-4-phenylbuto-xy]-1,9-phenanthridinediol 1-acetate. (−)-(6S,6aR,9R,10aR)-5,6,6a,7,8,9,10,10a-octahydro-6-methyl-3-[(R)-1-methyl-4-phenylbuto-xy]-1,9-phenanthridinediol 1-acetate is particularly useful in pain control, and its synthesis is described in Belgian Pat. No. 854,655, which is hereby incorporated by reference; in U.S. Pat. Nos. 4,206,225, 4,232,018, and 4,260,764, each to Johnson, which are hereby incorporated by reference; in U.S. Pat. No. 4,235,913 to Johnson et al., which is hereby incorporated by reference; in U.S. Pat. No. 4,243,674 to Bindra, which is hereby incorporated by reference; and in U.S. Pat. Nos. 4,263,438, 4,270,005, and 4,283,569, each to Althuis et al., which are hereby incorporated by reference.

“(−)-HU-210,” as used herein, is meant to refer to (−)-(3S,4S)-7-hydroxy-Δ⁶-tetrahydrocannabinol-1,1-dimethylhept-yl as well as to pharmaceutically acceptable salts, solvates, metabolites (e.g., cutaneous metabolites), and metabolic precursors of (−)-(3S,4S)-7-hydroxy-Δ⁶-tetrahydrocannabinol-1,1-dimethylhept-yl. (−)-(3S,4S)-7-hydroxy-Δ⁶-tetrahydro-cannabinol-1,1-dimethylh-eptyl is particularly useful in pain control, and its preparation is described in U.S. Pat. Nos. 4,876,276 and 5,521,215, each to Mechoulam et al., which are hereby incorporated by reference.

“(+)-HU-210,” as used herein, is meant to refer to (+)-(3S,4S)-7-hydroxy-Δ⁶-tetrahydrocannabinol-1,1-dimethylhept-yl as well as to pharmaceutically acceptable salts, solvates, metabolites (e.g., cutaneous metabolites), and metabolic precursors of (+)-(3S,4S)-7-hydroxy-Δ⁶-tetrahydrocannabinol-1,1-dimethylhept-yl. (+)-(3S,4S)-7-hydroxy-Δ⁹-tetra-hydrocannabinol-1,1-dimethylh-eptyl is sometimes referred to as HU-211 and/or dexanabinol; it is an antagonist of the N-methyl-D-aspartate receptor; and its preparation is described in U.S. Pat. Nos. 4,876,276 and 5,521,215, each to Mechoulam et al., which are hereby incorporated by reference.

“11-hydroxy-Δ⁹-THC,” as used herein is meant to refer to 11-hydroxy-Δ⁹-tetrahydrocannabinol as well as to its pharmaceutically acceptable salts, solvates, metabolites (e.g., cutaneous metabolites), and metabolic precursors. 11-hydroxy-Δ⁹-tetrahyd-rocannabinol is a more hydrophilic, psychoactive metabolite of Δ⁹-tetrahydrocannabinol, and its laboratory synthesis has been described in Siegel et al., J. Org. Chem., 54:5428 (1989), which is hereby incorporated by reference.

“Δ⁸-THC-11-oic acid,” as used herein, is meant to refer to Δ⁸-tetrahydrocannabinol-11-oic acid, as well as to its pharmaceutically acceptable salts, solvates, metabolites (e.g., cutaneous metabolites), and metabolic precursors. Δ⁸-tetrahydrocannabino-1-11-oic acid is a naturally occurring derivative of 6a,7,10,10a-tetrahydro-6,6,9-trimethyl-3-pentyl-6H-dibenzo[b,d]pyran-1-ol (which is a minor component of Cannabis sativa) and is produced from 6a,7,10,10a-tetrahydro-6,6,9-trimethyl-3-pentyl-6H-dibenzo[b,d]pyran-1-o-1 via a series of biotransformations mediated primarily by mammalian liver enzymes. A8-tetrahydrocannabinol-11-oic acid can also be produced synthetically by reference to the synthetic schemes set forth in U.S. Pat. No. 6,162,829 to Burstein, which is hereby incorporated by reference. Δ⁸-tetrahydrocannabin-ol-11-oic acid is more hydrophilic than 6a,7,10,10a-tetrahydro-6,6,9-trimethyl-3-pentyl-6H-diben-zo [b,d]pyran-1-ol, and it has analgesic activity.

“CP 55,940,” as used herein, refers to 4-(1,1-dimethylheptyl)-2,3′-dihydroxy-6′ alpha-(3-hydroxypropyl)-1′,2′,3′,4′,5′,6′-hexahydrobiphenyl, as well as to its pharmaceutically acceptable salts, solvates, metabolites (e.g., cutaneous metabolites), and metabolic precursors. 4-(1,1-dimethylheptyl)-2,3′-dihydroxy-6′ alpha-(3-hydroxypropyl)-1′,2′,3′,4′,5′,6′-hexahydro-biphenyl is sometimes referred to as (−)-cis-3-[2-Hydroxy-4-(1,1-dimethylheptyl)phenyl]-trans-4-(3-hydroxyprop-yl)cyclohexanol, and it is commercially available from Tocris Cookson, Inc., Ellisville, Mo. Its preparation has been described in U.S. Pat. No. 4,371,720 to Johnson et al. and U.S. Pat. No. 4,663,474 to Urban, which are hereby incorporated by reference.

“R(+)-WIN 55, 212-2,” as used herein, refers to (R)-(+)-[2,3-dihydro-5-methyl-3-(4-morpholinylmethyl)-pyrrolo[1,2,3-de]-1-,4-benzoxazin-6-yl]-1-naphthalenyl-methanone, as well as to its pharmaceutically acceptable salts, solvates, metabolites (e.g., cutaneous metabolites), and metabolic precursors. (R)-(+)-[2,3-dihydro-5-methyl-3-(−4-morpholinylmethyl)-pyrrolo[1,2,3-de]-1,4-benzoxazin-6-yl]-1-naphthalenyl-1-methanone (in its mesylate form) is commercially available, for example, from Tocris Cookson, Inc., Ellisville, Mo., and from Research Biochemicals International, Natick, Mass.

The cannabinoid-containing composition may further include one or more additional cannabinoids. The one or more additional cannabinoids can be selected from the aforementioned list of cannabinoids or it (they) can be selected from cannabinoids which are not contained in the aforementioned list, such as Δ⁸-THC, high affinity cannabinoid receptor agonists (other than R(+)-WIN 55, 212-2 and CP 55,940), and the like. Illustratively, the cannabinoid composition can include two or more cannabinoids, each being selected from the group consisting of Δ⁹-THC, cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2.

“Metabolic precursors” of cannabinoids, as used herein, are meant to include prodrugs and other materials that are metabolized in the subject's body (e.g., cutaneously or systemically or both) to a cannabinoid or an active cannabinoid mimetic. Suitable metabolic precursors include those that are less lipophilic (i.e., more water soluble) relative to the cannabinoid into which they are metabolized. Examples of such metabolic precursors include those described in, for example, U.S. Pat. No. 5,847,128 to Martin et al., which is hereby incorporated by reference.

In one embodiment, illustrative cannabidiol prodrugs include those compounds of Formula (I):

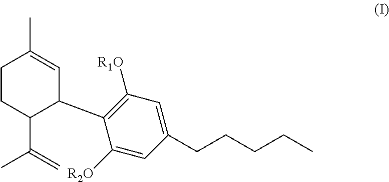

wherein

R₁ and R₂ can be the same or different and are each independently comprised of a hydrogen and/or a bio-labile linker (e.g. ester, oxygenated ester, oxaester, pegylated ester, hydroxylated ester, alkyl ester, amino ester, alkylamino ester, dialkylamino ester, carbonate, alkyl carbonate, carbamate, alkyl carbamate, amino carbamate, alkylamino carbamate, dialkylamino carbamate, or other suitable bio-labile linking structure) and further comprising moieties which can be selected in order to control the rate and extent of absorption and metabolism, including transdermal absorption and metabolism. However, R₁ and R₂ cannot both be a hydrogen atom. Several options for R₁ and R₂ are disclosed herein. Also included herein is the free base, salt, ester, hydrate, amide, enantiomer, isomer, tautomer, polymorph, or derivative thereof of compounds of Formula I.

In a further embodiment, the cannabidiol prodrug can be selected from a group comprising:

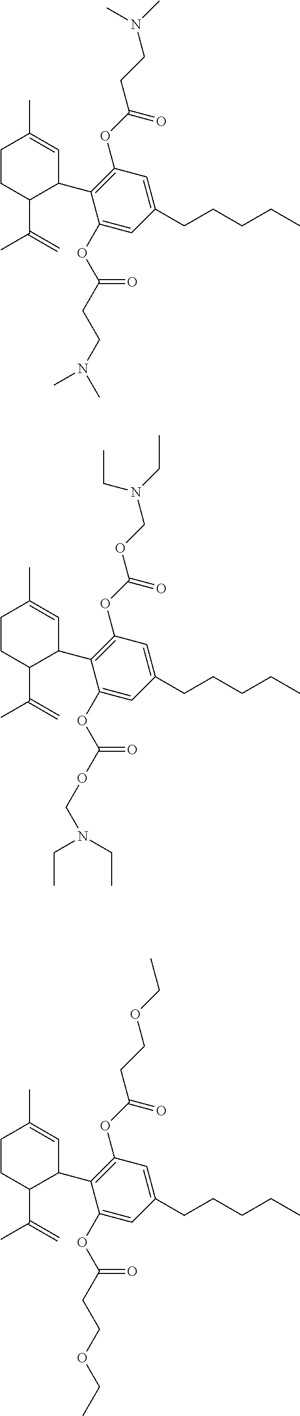

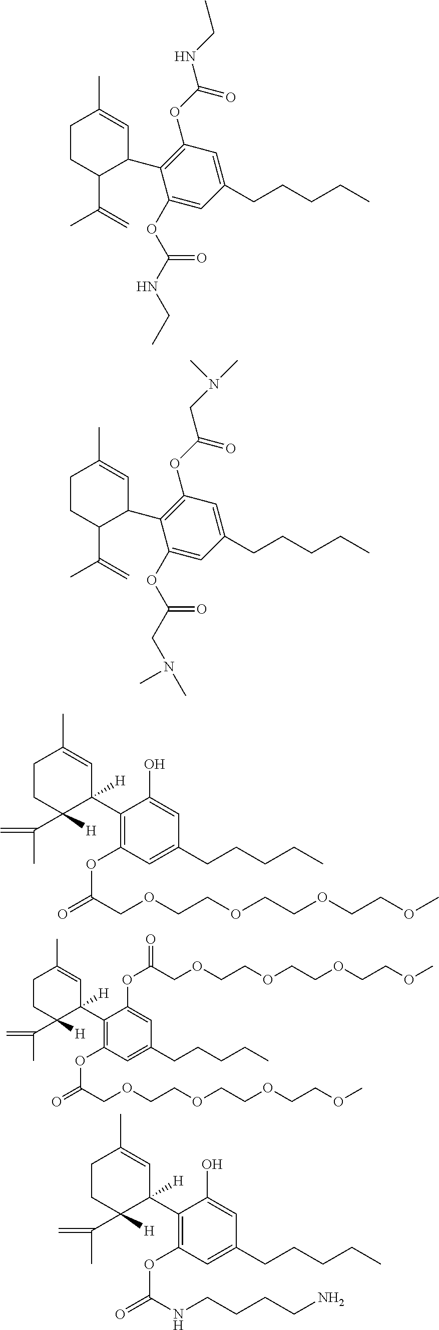

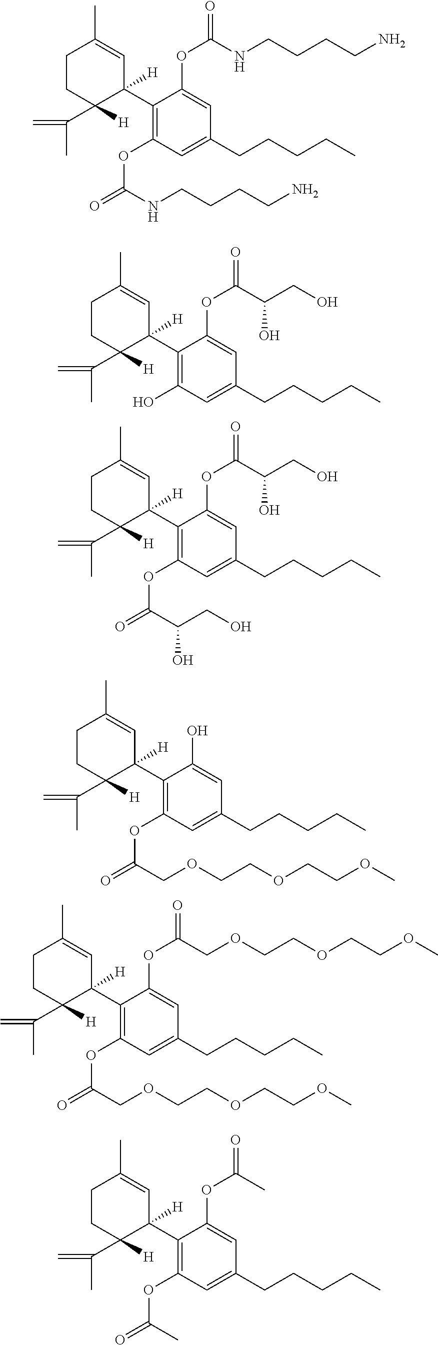

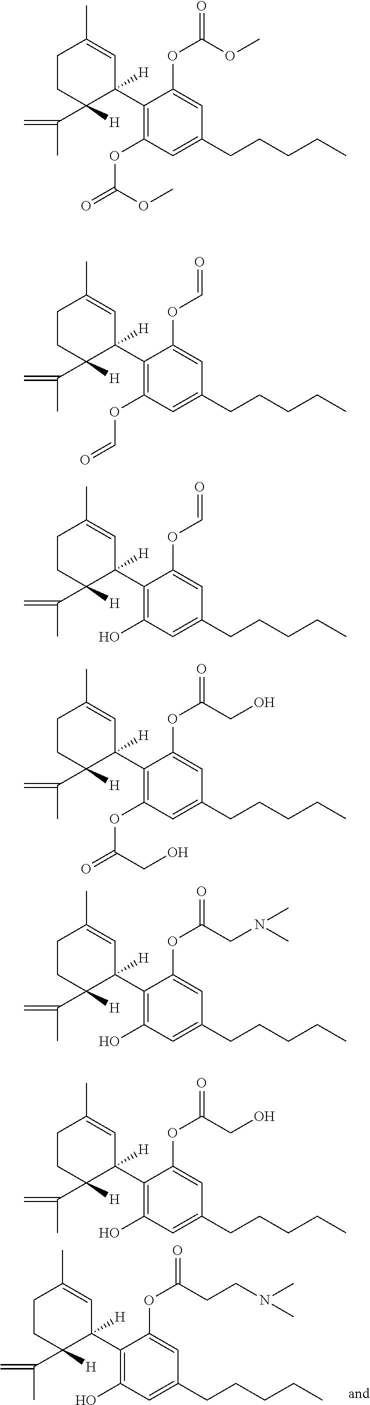

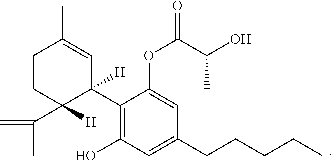

In a further embodiment, one or more cannabidiol prodrug can be used with or instead of cannabidiol or other cannabinoids in the pharmaceutical compositions described herein. In an additional embodiment, a cannabidiol prodrug can be used with or instead of cannabidiol or other cannabinoids in the method of administering cannabidiol to mammal described herein. In an another embodiment, a cannabidiol prodrug can be used with or instead of cannabidiol or other cannabinoids in the method of treating a medical condition by the administration of cannabidiol described herein, wherein the medical condition is selected from a group consisting of nausea, vomiting, emesis, pain, wasting syndrome, HIV-wasting, chemotherapy induced nausea and vomiting, alcohol use disorders, dystonia, multiple sclerosis, inflammatory bowel disorders, arthritis, dermatitis, Rheumatoid arthritis, systemic lupus erythematosus, anti-inflammatory, anti-convulsant, anti-psychotic, antioxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, synovitis, juvenile rheumatoid arthritis and inhibition of hair growth.

“Metabolites” of cannabinoids, as used herein, are meant to include compounds which are produced by the metabolic processes (e.g., cutaneous metabolic processes and/or systemic metabolic processes) of the subject's body. Suitable metabolites can be identified, for example, by studying the kinetics of drug enzymatic metabolism in skin homogenates. Illustratively, skin homogenates can be prepared from 250-μm dermatomed fresh healthy abdominal plastic surgery samples. The skin is homogenized (e.g., using a Polytron tissue homogenizer and ground glass homogenizer fitted with a glass pestle) in 4-(2-hydroxyethyl)-1-piperazineethanesulfonic acid (“HEPES”)-buffered Hanks' balanced salt solution. Whole homogenates can be used for these studies or, if significant mitochondrial or nuclear metabolism is found not to occur (e.g., by comparing the degree of metabolism in the supernatant the degree of metabolism in the whole homogenate), the studies can be carried out on only the supernatant fraction. The drug (solubilized in, for example, buffer, ethanol, dimethylsulfoxide, or combinations thereof) is then incubated with the homogenate (or supernatant) along with NADPH (or a generating system), NADH, MgCl₂, and bovine serum albumin. The total volume of ethanol in the reaction mixture should be small (e.g., under 2%) to help minimize ethanol's detrimental effects on the enzymes. After incubating for a period of time, the reaction is terminated with 15% trichloroacetic acid, and the drug and its metabolites are obtained by solid-phase extraction. The metabolite or metabolites formed can then be identified and assayed by any suitable method (e.g., HPLC).

The term “therapeutically effective amount” or “therapeutically and/or prophylactically effective amount” as used herein refers to an amount of a cannabinoid that is sufficient to elicit the required or desired therapeutic and/or prophylactic response. Preferably the “therapeutically effective amount” or “therapeutically and/or prophylactically effective amount” of cannabinoid is sufficient to alleviate the symptoms associated with osteoarthritis.

It is understood that a therapeutically and/or prophylactically effective amount of a drug for a subject is dependent inter alia on the body weight of the subject as well as other factors known to a person of ordinary skill in the art. A “subject” herein to which a therapeutic agent or composition thereof can be administered includes mammals such as a human subject of either sex and of any age, and also includes any nonhuman animal, particularly a domestic, farm or companion animal, illustratively a mouse, a cat, cow, pig, dog or a horse as well as laboratory animals such as guinea pigs and primates.

As used herein, the term “gel”, “gel-like” or “gel matrix” means a type of reservoir or vehicle for the cannabinoid. A gel takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles), or a network of polymer chains. In some embodiments, a gel matrix may include hydrogels, organogels, and the like. The terms gel, gel-like and gel matrix also include compositions comprised of polyacrylic acid polymers which have been partially neutralized by use of a neutralizing agent in order to increase the viscosity of the compostion.

A “hydrogel” or “hydrogel matrix” refers to a three dimensional network of, for example, cross-linked hydrophilic polymers in the form of a gel and substantially composed of water.

Protocols for forming gels, including hydrogels, are well known in the art, as are protocols for forming gels and hydrogels comprising therapeutically effective amounts of one or more active agents.

As one skilled in the art will recognize, optimization of the method of the present invention will involve consideration of a variety of factors in selecting the cannabinoid to be used. One such factor is skin permeability. Several physicochemical factors influence the ability of cannabinoids to penetrate the skin. These include the cannabinoid's molecular weight, its molecular volume, its lipophilicity, its hydrogen bonding potentials, its polarity, etc. as well as the use of a penetration enhancer or combination of multiple penetration enhancers.

As indicated above, the cannabinoid is applied to the surface of the skin of a mammal for transdermal or topical delivery of the cannabinoid to the subject, for example, by applying the cannabinoid or cannabinoid-containing composition to the subject's skin and rubbing the composition into the subject's skin for absorption of the cannabinoid. Generally transdermal delivery involves contacting the cannabinoid or cannabinoid-containing composition with the subject's skin under conditions effective for at least one of the provided cannabinoids to penetrate the skin.

The cannabinoid-containing composition can be formulated as a transdermal or topical gel formulation. The transdermal and topical gel formulations can include inert diluents and carriers as well as other conventional excipients, such as wetting agents, preservatives, and suspending and dispersing agents. In addition to the above, generally non-active components, topical gel formulations containing the cannabinoid can further include other active materials, particularly, active materials which have been identified as useful in the treatment of pain, discomfort, or other conditions associated with a subject's illness and which can usefully be delivered transdermally to the subject. For instance, such other active materials can include analgesics, such as opiates and other analgesic active materials which operate on non-cannabinoid receptors. Where, for example, opiates are included, transdermally deliverable opiates are particularly preferred. One example of a transdermally deliverable opiate is fentanyl. The topical formulation can be applied directly to the skin until it is completely or almost completely absorbed into the skin. The gel may be optionally covered (e.g., with a bandage of gauze) to minimize the likelihood of its being disturbed. Alternatively, the topical gel formulation can be coated on the surface of a bandage, gauze, etc., and the bandage, gauze, etc. can then be applied to the skin of the subject such that the topical gel formulation is in direct contact with the subject's skin. Preferably, the topical gel is not coated onto the surface of a bandage or gauze, etc. for application to the subjects skin.

The cannabinoid composition can also include one or more inhibitors of cannabinoid metabolism, particularly in cases where inhibition of cutaneous metabolism is needed to increase therapeutic drug levels. Such inhibitors of cannabinoid metabolism can include inhibitors of the P450 enzymes or other identified critical enzymatic processes. Suitable inhibitors of cannabinoid metabolism include, for example, essential oils which inhibit the activity of cytochrome P450 3A in the skin, such as those described in U.S. Pat. No. 5,716,928 to Benet et al., which is hereby incorporated by reference. Some of these essential oils may also act as transdermal penetration enhancers, thus providing a dual mechanism of percutaneous penetration increase.

The present invention is further illustrated with the following examples.

### EXAMPLES

### A. Materials and Methods

### 1. Materials

CBD was a generous gift obtained from National Institute on Drug Abuse (“NIDA”). CFA was obtained from DIFCO Laboratories (Detroit, Mich.). Isopropyl myristate (IPM), sodium hydroxide, ethyl acetate (HPLC grade), and ammonium acetate (HPLC grade) were purchased through Fisher Scientific (Fairlawn, N.J.). Acetonitrile (ACN) (HPLC grade) was purchased from VWR (West Chester, Pa.). Absolute ethanol (USP grade) was purchased from Sigma-Aldrich (St. Louis, Mo.). Pre-purified nitrogen was purchased from Scott-Gross Company Inc (Lexington, Ky.). Carbopol® 980 was obtained from Noveon, Inc. (Cleveland, Ohio). Nanopure water was obtained from a Barnstead NANOpure® Dlamond™ ultrapure water filtration system (Dubuque, Iowa).

### 2. Gel Preparation

Gels with and without 1% w/w or 10% w/w CBD were prepared in a similar manner. The respective amount of CBD was weighed and dissolved in 72.5% w/w ethanol. Once dissolved, 20.5% w/w nanopure water was added to the solution to and then isopropyl myristate (IPM) 0.5% w/w was added. Carbopol™ 980 0.9% w/w was added to the solution and then sonicated for 10 min to ensure complete incorporation of the Carbopol™ 980. Sodium hydroxide (0.1 N solution) 4.7% w/w was added to the sonicated solution to initiate thickening of the gel. The formulation was then sonicated for an additional 10 min, loaded into 1 mL syringes and sealed. The gels were made prior to the initial dosing and the pre-filled syringres were stored at ambient temperature in the dark for the entire week of application. The stability of the CBD containing gel at room temperature over 8 days was evaluated. No degradation was observed in the concentration of CBD in the gel. The CBD-containing gel failed to display any visible changes over the course of 8 days. The gel with and without CBD remained clear and colorless.

### 3. Animals

All animal procedures were approved by the University of Kentucky IACUC committee.

Experiments were performed using Sprague-Dawley male rats (260-280 g) purchased from Harlan Sprague Dawley, Inc. (Indianapolis, Ind.). Rats were housed in individual cages and allowed access to food and water ad libitum except during testing and dosing in a quiet room with a 12 h/12 h dark/light cycle.

### 4. Induction of Arthritis

To induce monoarthritis, rats were briefly anesthetized by isoflurane inhalation; the left knee joint cavity was injected with 0.1 mL of 2 mg/mL suspension of Mycobacterium tuberculosis (CFA) in 1:1 peanut oil and saline. Rats were monitored daily and knee joint inflammation and pain were assessed before CFA injection and daily beginning on day 3 after CFA (days 3-7).

### B. Assessment of Knee Joint Inflammation and Pain-Related Posture

1. Measurement of the Knee Joint Circumference

The circumference of the affected knee joints were measured in cm with a flexible tape wrapped around the center of the joint while the hind limbs were held in extension both before injection of CFA and after 4 and 7 days following the injection of CFA. The knee joint circumferences of rats treated with the CBD containing gel and the gel without CBD (the “vehicle gel”) were measured and compared (see Table 1).

The results presented in Table 1 demonstrate that the knee joint circumferences (cm) due to CFA induced inflammation were significantly decreased for rats receiving the 6.2 mg/d (6.6±0.2) and 62.3 mg/d (6.6±0.1) CBD doses (p<0.05) compared to those of the rats given vehicle gel respectively (7.2±0.3 and 7.0±0.0) (Table 1). Normal knee joint circumferences measured for each group prior to receiving vehicle gel or CBD were 5.8±0.2 and 5.7±0.2. There were no significant differences (p>0.05) in knee joint temperature among the treatment groups (Table 2).

TABLE 1
Assessment of CFA-induced knee joint inflammation after the
application of CBD or vehicle gel for four consecutive days (day 4)
Knee joint circumference (cm) [mean ± SD]

| | | CFA + PLA | CFA + CBD |
|---|---|---|---|
| Baseline | | 5.8 ± 0.2^{b} | 5.7 ± 0.2^{c} |
| 0.6 mg/d | CFA | 7.6 ± 0.2^{a} | 7.5 ± 0.3^{b} |
| | Post-CBD | 7.1 ± 0.2^{a} | 7.0 ± 0.3^{b} |
| 3.1 mg/d | CFA | 7.8 ± 0.2^{a} | 7.4 ± 0.4^{b} |
| | Post-CBD | 7.2 ± 0.4^{a} | 7.1 ± 0.3^{b} |
| 6.2 mg/d | CFA | 7.4 ± 0.4^{a} | 7.2 ± 0.3^{b} |
| | Post-CBD | 7.2 ± 0.3^{a} | 6.6 ± 0.2^{b}* |
| 62.3 mg/d | CFA | 7.1 ± 0.1^{a} | 7.2 ± 0.0^{a} |
| | Post-CBD | 7.0 ± 0.0^{a} | 6.6 ± 0.1^{a}* |

*p < 0.05
^{a}n = 3;
^{b}n = 5;
^{c}n = 8

TABLE 2
Assessment of CFA-induced knee joint temperature after the
application of CBD or vehicle gel for four consecutive days (day 4)
Knee joint temperature (° C.) [mean ± SD]

| | | | CFA + |
|---|---|---|---|
| | | CFA + PLA | CBD |
| Baseline | | 33.6 ± 1.1^{a} | 33.4 ± 0.2^{a} |
| 0.6 mg/d | CFA | 32.8 ± 0.4^{a} | 31.7 ± 1.7^{b} |
| | Post-CBD | 31.4 ± 0.2^{a} | 31.2 ± 0.7^{b} |
| 3.1 mg/d | CFA | 31.9 ± 0.8^{a} | 30.8 ± 0.9^{b} |
| | Post-CBD | 30.9 ± 1.4^{a} | 30.6 ± 0.6^{b} |
| 6.2 mg/d | CFA | 33.9 ± 0.3^{a} | 32.1 ± 1.8^{b} |
| | Post-CBD | 33.1 ± 0.4^{a} | 31.7 ± 2.1^{b} |
| 62.3 mg/d | CFA | 33.5 ± 0.6^{a} | 34.1 ± 0.8^{a} |
| | Post-CBD | 31.0 ± 1.1^{a} | 30.2 ± 0.9^{a} |

^{a}n = 3;
^{b}n = 5

2. Evaluation of the Pain-Related Posture

The abnormal posture of the affected hind limb of each animal was assigned a single score using a subjective pain-related behavioral scale (spontaneous pain rating score 0-5), i.e. 0—normal; 1—curling of the toes, 2—eversion of the paw; 3—partial weight bearing; 4—non-weight bearing and guarding; and 5—avoidance of any contact with the hindlimb (Sluka et al. (1994) Neuroreport 5:109-112).

Pain scores after 6.2 mg/d (1.6±0.5) and 62.3 mg/d (1.7±0.6) doses in CFA post-CBD treated rats were improved (p<0.05) compared to their respective CFA control rats (4.0±0.0 and 3.7±0.6) (Table 3). Pain scores for CFA post-CBD rats 0.6 mg/d (4.0±0.0) and 3.1 mg/d (3.7±0.3) were similar compared to their respective CFA control rats (4.0±0.0 and 3.5±0.0). Normal rats score 0 in this test.

TABLE 3
Assessment of CFA-induced pain rating scores after the application
of CBD or vehicle gel for four consecutive days (day 4)
Spontaneous pain rating scores [mean ± SD]

| | | CFA + PLA | CFA + CBD |
|---|---|---|---|
| 0.6 mg/d | CFA | 4.0 ± 0.0^{a} | 4.0 ± 0.0^{b} |
| | Post-CBD | 4.0 ± 0.0^{a} | 4.0 ± 0.0^{b} |
| 3.1 mg/d | CFA | 4.0 ± 0.0^{a} | 4.0 ± 0.0^{b} |
| | Post-CBD | 3.5 ± 0.0^{a} | 3.7 ± 0.3^{b} |
| 6.2 mg/d | CFA | 4.0 ± 0.0^{a} | 4.0 ± 0.0^{b} |
| | Post-CBD | 4.0 ± 0.0^{a} | 1.6 ± 0.5^{b}* |
| 62.3 mg/d | CFA | 4.0 ± 0.0^{a} | 4.0 ± 0.0^{a} |
| | Post-CBD | 3.7 ± 0.6^{a} | 1.7 ± 0.6^{a}* |

*p < 0.05
^{a}n = 3;
^{b}n = 5

### C. Assessment of Pain Related Behaviors

1. Secondary Thermal Hyperalgesia

Secondary thermal hyperalgesia was assessed with the paw withdrawal latency (PWL) test (Hargreaves et al. (1988) Pain 32: 77-88). PWLs were measured in response to a radiant heat source shone on one hind paw of the animals from beneath a glass top table. A shortened PWL response in animals with knee joint inflammation was indicative of secondary thermal hyperalgesia. Animals were placed in separate plastic cubicles (4×4×10 in) on a glass top table (2 mm thickness of glass) and allowed to adapt to their new environment for 20-30 min before testing. A small movable metal box that focused a high-intensity light beam through an aperture (0.5 cm×0.5 cm) was attached to an on/off switch and a digital timer. The light beam was applied to the plantar surface of the hind paw through the glass until the rat lifted its paw. At that time, the light is switched off and the number of seconds on the digital timer recorded as the PWL (sec). The maximal cut off time for the paw-withdrawal reflex was set to 15 sec (55° C.). Thereafter, PWL tests were performed 4 h after CBD application for the consecutive 4 days. Both hind paws were tested independently for 5 trials per side with 5-minute intervals between trials. A mean of the 5 trials was calculated for each animal. The PWL tests were performed before knee joint injection as a baseline and 5 times after the CFA injection beginning on day 3 (days 3-7). The examiner was blind from the treatment group of animals.

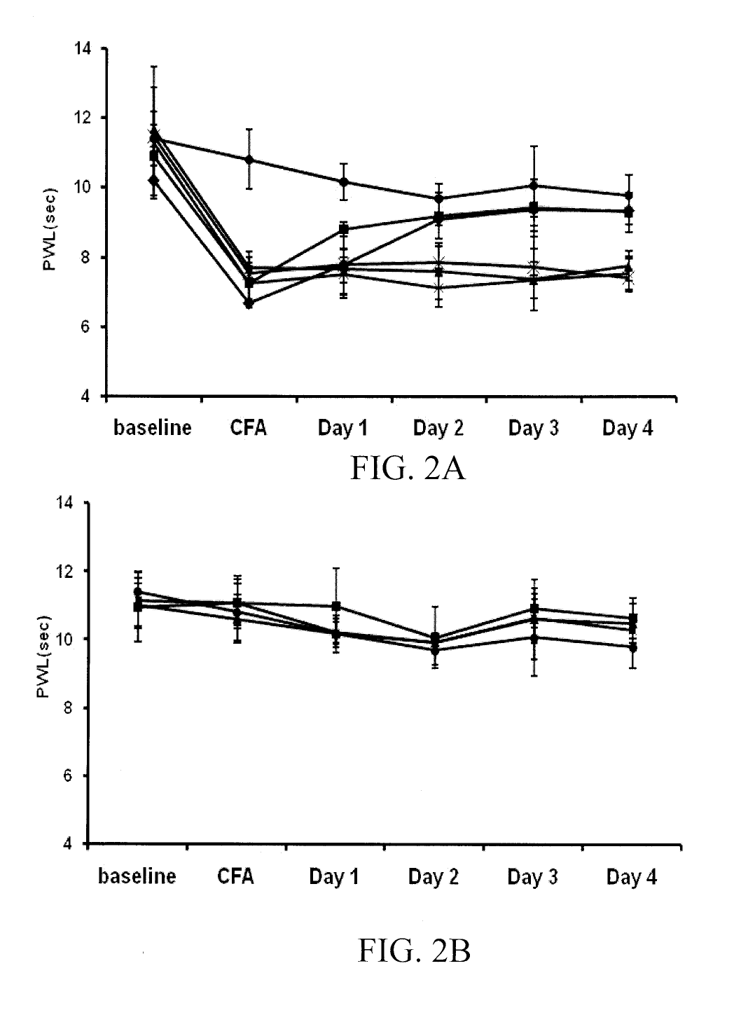

PWLs (sec) performed on the opposite uninflamed (right) paw of mono-arthritic rats showed no differences in baseline across all treatment groups. All CFA rats demonstrated a good response to the CFA, and they had significantly shortened (p<0.05) PWL for the inflamed paw compared to the normal vehicle injected rats. By day 2 of CBD application, a significant improvement (p<0.05) for the 6.2 mg/d (9.2±0.7) and 62.3 mg/d (9.1±0.2) CBD treatments was observed as compared to vehicle gel (7.1±0.6), 0.6 mg/d (7.8±0.5), and 3.1 mg/d (7.6±0.8) treatments (FIG. 2A). There were no significant differences (p>0.05) between the 6.2 mg/d and 62.3 mg/d treatments on day 2. The PWL scores for the 6.2 mg/d and 62.3 mg/d doses were similar to those of the normal vehicle treated rats (9.7±0.4) by the second day of cannabidiol gel application. On day 2, normal rats receiving the vehicle gel (9.7±0.4), or 0.6 mg/d (9.9±0.2), 3.1 mg/d (9.9±0.3), and 6.2 mg/d (10.1±0.9) CBD, all had similar PWL values indicating that the CBD had no effect in normal rats (FIG. 2B). By day 4, CFA rats receiving 0.6 mg/d and 3.1 mg/d CBD still did not have improved PWL scores. The day 4 scores for CFA rats receiving 6.2 mg/d and 62.3 mg/d were equivalent to each other (9.2±0.3 and 9.3±0.2) and to their own baseline scores (10.9±0.3 and 9.3±0.3). In fact, the day 4 values for the normal controls that received vehicle gel (9.4±0.6) and normal controls that received 6.2 mg/d CBD gel (10.6±0.3) were not different.

2. Open Field Behavioral Testing

Exploratory behavioral activity of the rats was monitored using the Flexfield Animal Activity System (San Diego Instruments; San Diego, Calif.) with a Photobeam Activity System software coupled to a computer according to previously published methods by Zhang et al., (2004) Pharmacol Biochem Behav 77: 145-153. Briefly, six main parameters were recorded: rearing events and rearing time, active time and resting time, total activity (i.e. number of photobeams disturbed in the x-y-z planes while the rat was moving around in the box or involved in stationary movement such as grooming), and distance traveled. Activity testing was conducted in an isolated, temperature controlled area at similar times daily in order to provide consistent results.

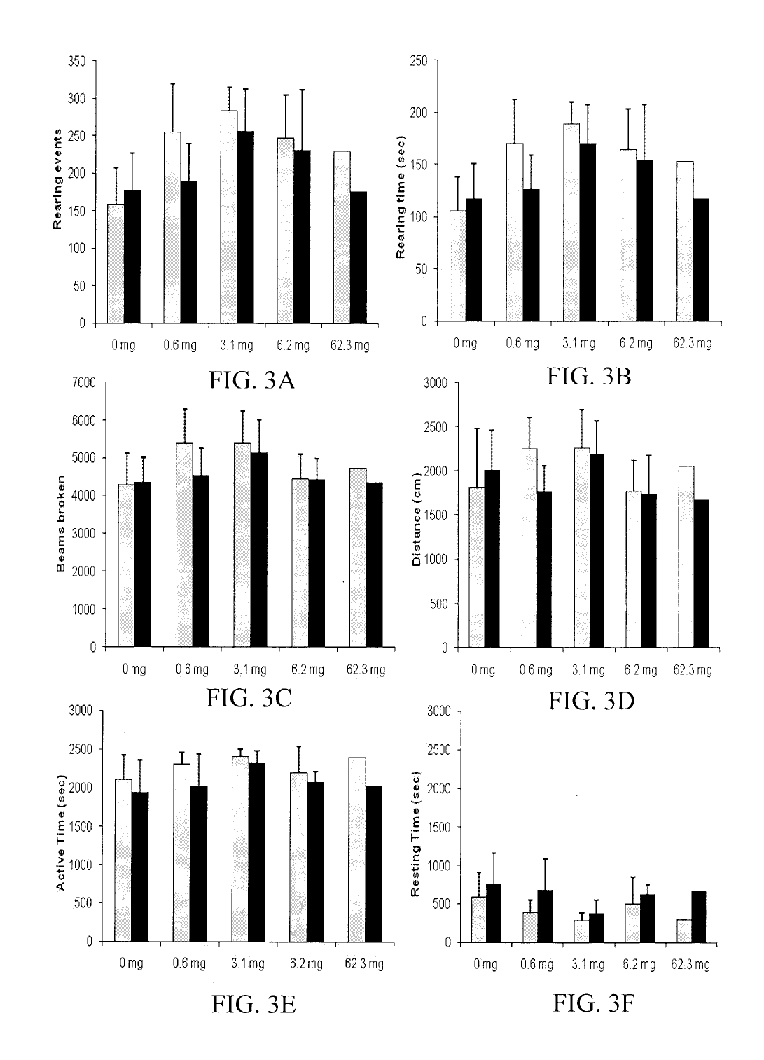

The exploratory activities in normal (non-CFA) rats receiving vehicle gel with or without CBD were assayed and compared. No differences (p>0.05) were determined among the groups for the six parameters except for a difference (p<0.05) between normal rats receiving only vehicle gel (158.3±49.2, 105.4±32.8) and normal rats receiving a 3.1 mg/d CBD (284.0±31.1, 189.1±20.7) treatment for pre-dose rearing events and rearing time, respectively. No significant (p>0.05) differences were seen when comparing the pre-dose values to the post-dose values among the five treatment groups for the six parameters: rearing events and time, total beam broken, distance traveled, active time, and resting time (FIGS. 3A-3F) except for distance traveled (cm) (p<0.05) with the 0.6 mg/d (pre-dose 2245.5±367.4 compared to post-dose 1757.1±299.2).

### D. Experimental Procedures

### 1. Cannabidiol Administration

Baseline parameters (body weight, knee joint temperature, and knee joint circumference) were obtained before the knee injection of rats with CFA. PWL test and exploratory activity testing were also conducted prior to CFA injection. Rats were anesthetized with a short term anesthetic and the left knee joint cavity was injected with CFA for induction of mono-arthritis. On day 3, the back of each animal was shaved. On day 4 or 7 following mono-arthritis induction, the following parameters (body weight, knee joint temperature, and knee joint circumference) were again obtained prior to initial skin application of CBD gel or vehicle gel. Rats received daily treatments for four consecutive days of either vehicle gel, or CBD gel. Gel was applied to the skin in a templated region on the dorsal surface of the rat and rubbed into the skin for 30 sec to ensure complete coverage. The nitrile template was used to ensure specific area reproducibility. The PWL test was conducted 4 h after gel application. Exploratory activity was monitored prior to initial gel application and on the third day post gel application (after 6 h). On the final day of gel application after the body and knee joint parameters were obtained and PWL test was conducted, rats were euthanized by pentobarbital overdose. Blood samples were obtained for CBD plasma quantification, and tissues (knee joints, dorsal root ganglia, spinal cord, brain) were collected after perfusion.

### 2. Experimental Groups

Half of the treated rats were injected with the CFA in the knee joint. Within each group of rats, half received vehicle gel without CBD and the other half received the gel CBD. Four different doses were tested in the rats, 0.6 mg/d, 3.1 mg/d, 6.2 mg/d, or 62.3 mg/d. Doses of CBD containing gel were based on previous studies with a 1% gel in hairless guinea pigs. Rats were initially dosed with CBD at 6.2 mg/d. A two-fold lower dose and a ten-fold lower/higher dose (3.1 mg/d, 0.6 mg/d, and 62.3 mg/d) were also investigated. Area of application, amount of gel, and/or concentration of gel was adjusted to achieve the desired doses. The areas of application and amount of gel applied correspond to the previously mentioned doses: 3.5 cm² (75 μL), 17.5 cm² (375 μL), 35.0 cm² (750 μL of 1% w/w CBD gel), and 35.0 cm² (750 μL of 10% w/w CBD gel).

### 3. Plasma Extraction

50 μL aliquot of plasma collected from cannabidiol dosed rats was added to a siliconized microcentrifuge tube containing 500 μL of 1:1 ACN:ethyl acetate. The plasma/ACN:ethyl acetate sample was vortexed for 30 sec and centrifuged at 10,000×g for 20 min. The supernatant was removed and placed into a clean silanized culture tube and evaporated under nitrogen in a 37° C. water bath. The dried sample was reconstituted with 100 μL of ACN and vortexed for 30 sec, then sonicated for 5 min. The sonicated sample was then placed into an HPLC vial containing a silanized low volume insert and the concentration of CBD in the plasma samples analyzed by LC/MS. Standards were prepared by spiking blank plasma samples with respective standard concentrations and extracting the standards similarly to the collected plasma samples.

The plasma CBD concentrations in rats receiving topical application of each of the four doses, i.e., 0.6 mg/d, 3.1 mg/d, 6.2 mg/d and 62.3 mg/d, were 3.8±1.4 ng/mL (n=9), 17.5±4.4 ng/mL (n=8), 33.3±9.7 ng/mL (n=8), and 1,629.9±379.0 ng/mL (n=4), respectively. Plasma concentrations of CBD from rats dosed with 0.6 mg/d, 3.1 mg/d and 6.2 mg/d displayed an excellent linear pharmacokinetic correlation (slope=1.0, R2=0.999). However, the 62.3 mg/d dose applied did not follow the linear pharmacokinetic profile.

### 4. Analytical LC/MS Method

The LC/MS system used to analyze samples was comprised of a Waters Alliance 2695 pump and autosampler, a Micromass™ ZQ detector, and 996 photodiode array detector with MassLynx™ software (Waters Corp., Milford, Mass.). A Symmetry™ C18 column (150×2.1 mm, 5 μm) with a Sentry Symmetry™ guard column (10×2.1 mm, 3.5 μm) was used with the LC/MS system. The ZQ detector was used with an electrospray ionization (ESI) probe set for single ion monitoring (SIM) for cannabidiol quantification. Analysis was performed in negative mode for m/z 313 [CBD-H]⁻ (dwell time: 30 sec). Capillary and cone voltage were set at 35 kV and 40 V, respectively. Source block and desolvation temperatures were set at 120° C. and 250° C., respectively. Nitrogen produced from the Nitroflow Lab (Parker Hannifin Corp; Cleveland, Ohio) was used as nebulization and drying gas flow rates of 50 and 450 L/h, respectively. Volume injected onto the column was 20 μL and run time was 10 min. The mobile phase was comprised of 75:25 ACN:2 mM ammonium acetate buffer w/5% ACN and used at a flow rate of 0.25 mL/min. Retention time for cannabidiol was 5.6-5.7 min. Standard curves were linear within the range of 2 ng/mL-300 ng/mL.

### 5. Data Analysis

Data are presented as mean±SD in tables and figures. Results were analyzed with Student paired t-test and one way ANOVA followed by Tukey post-hoc analysis to determine if there were significant differences before and after treatment and among treatment groups. SigmaStat™ 2.03 was the statistical program used to calculate results (Systat™ Software, Inc.; Richmond, Calif.). Values of p<0.05 were considered significant.

### E. Discussion

### 1. Transdermal CBD Correlation to Blood Absorption

Plasma concentrations of CBD from rats dosed with 0.6 mg/d, 3.1 mg/d and 6.2 mg/d exhibited an excellent linear correlation; however, the 62.3 mg/d dose did not fit into the linear pharmacokinetic profile. Without wishing to be bound by theory, the failure of the 62.3 mg/d dose to fit into the profile could be due to a change in the absorption (from the formulation change in dose per unit area, drug saturation level in the vehicle, and/or change in partitioning), and/or the achievement of capacity-limited metabolism at this large dose. With the 62.3 mg/d dose, the 10% gel formulation was very close to solubility saturation, which may have caused an increased absorption rate, as compared to the 1% formulations used. In a study by Paudel et al. (unpublished) in hairless guinea pigs dosed with a 1% CBD gel, contact studies were performed to determine the amount of residual drug left on the surface of the skin. Results showed that approximately 15% residual drug remained on the skin surface up to 8 h post application of a hydroalcoholic CBD gel. Higher concentration CBD gels were not examined. Due to the limited surface area available to topically dose a rat, the area for the 6.2 mg/d and 62.3 mg/d doses were the same. Also with 62.3 mg/d, especially given the size of the rat (260-280 g), systemic CBD levels may have been higher than predicted by a linear pharmacokinetic relationship if a zero-order elimination process was reached at this high dose. Paudel et al (unpublished) saw a Cₘₐₓ plasma concentration of 45.8±8.4 ng/mL (n=3) in hairless guinea pigs dosed once with the 6.2 mg/d CBD gel compared to 85.6±48.4 ng/mL (n=5) after 3 consecutive days of gel application. The Tₘₐₓ for a single application to the hairless guinea pigs was 6.7±2.1 h compared to 40.0±14.5 h after multiple applications. CBD plasma concentrations were detected out to 120 h after a single initial gel application. The results presented herein indicate that topical administration of CBD to an afflicted area in osteoarthritis is likely to improve pain and inflammation relief by providing a higher tissue concentration of drug at the site of injury.

### 2. Assessment of Knee Joint Inflammation and Nociceptive Responses

Knee Joint Inflammation. A normal knee circumference of a 250 g rat was 5.6 cm. Reduction in the knee joint inflammation has been seen previously with orally administered cannabidiol treatments (Costa et al. (2007) Eur J Pharmacol 556: 75-83). The results presented herein demonstrate that the 6.2 mg/d dose appeared to be the optimal treatment since no further improvement in the knee inflammation was observed with the 62.3 mg/d treatment. A reduction in inflammation is important for the symptomatic treatment of osteoarthritis because osteophyte formation leads to enlargement of the affected joints reducing physical functioning and increasing pain (Buckwalter and Martin, (2006) Adv Drug Deliv Rev 58: 150-167; Jones et al. (2000) J Rheumatol 27: 745-752. No knee joint temperature differences were measured among treatment groups. Barak et al. (1992) Biotherapy 4: 317-323 and Lu et al. (2008) Eur J Neurosci 27:1153-1165 had similar results for other agents that reduced inflammation with no temperature differences measured between control groups and treated groups of rats using the CFA arthritis model.

Spontaneous Pain Rating Scores. Pain caused by osteoarthritis is typically the reason individuals first seek medical treatment. A successful treatment for osteoarthritis should not only reduce inflammation but also eliminate pain. Spontaneous pain related posture scores (scale 1-5) improved (p<0.05) for CFA rats treated with CBD 6.2 mg/d (pre-dose 4.0±0.0 to post-dose 1.6±0.6) and 62.3 mg/d (pre-dose 4.0±0.0 to post-dose 1.7±0.6) compared to their respective CFA controls receiving vehicle gel (pre-dose 4.0±0.0 to post-dose 4.0±0.0 and pre-dose 4.0±0.0 to post-dose 3.7±0.6). The 6.2 mg/d and 62.3 mg/d treatments were similar in their reduction of pain scores.

Secondary Thermal Hyperalgesia. An improvement in PWL was seen in the 6.2 mg/d and 62.3 mg/d doses. However, the improvement in PWL was similar between the 6.2 mg/d dose and 62.3 mg/d. Malfait et al. (2000) (Proc Natl Acad Sci USA 97: 9561-9566) reported similar results in mice with collagen-induced arthritis, a rheumatoid arthritis model, treated with cannabidiol either i.p. or orally. The highest treatment dose of CBD administered also did not perform as well as a lower treatment in their studies. Of the i.p. treatments they administered (2.5 mg/kg, 5 mg/kg, 10 mg/kg, or 20 mg/kg), 5 mg/kg was optimal for arthritis suppression in mice and for orally administered treatments (10 mg/kg, 25 mg/kg or 50 mg/kg), 25 mg/kg was optimal (Malfait et al. (2000)). Costa et al. (2004a) Naunyn-Schmiedebergs Arch Pharmacol 369: 294-299 saw vast improvements in PWL in mice with 10 mg/kg, 20 mg/kg, and 40 mg/kg oral CBD administration at 3 h post carrageenan-induced paw edema with the elimination of hyperalgesia. Even by 6 h, the lower two doses administered orally, 5 mg/kg and 7.5 mg/kg, had eliminated hyperalgesia. For comparison, the transdermal doses used for the current studies would have been approximately 2.3 mg/kg, 11.5 mg/kg, 23.0 mg/kg, and a 230.0 mg/kg dose. The results presented herein demonstrate an improvement (p<0.05) in PWL for rats with CFA induced mono-arthritis at 4 h (d 1) post CBD dosing with the 6.2 mg/d dose compared to the CFA rats receiving vehicle gel. The improvement continued for the duration of the study. By day 2, no differences (p>0.05) in PWL were seen between the normal rats and rats receiving 6.2 mg/d and 62.3 mg/d transdermal CBD.

### 3. Assessment of Psychoactive Effects

Exploratory Behavioral Activity. During exploratory behavioral testing, no differences in activity measurements were determined in normal rats between pre-CBD and after four days of CBD administration except for distance traveled (cm) (p<0.05) with the 0.6 mg/d (pre-dose 2245.5±367.4 compared to post-dose 1757.1±299.2). CBD is a known non-psychoactive drug, and due to its low affinity for the CB1 receptor (Croxford (2003) CNS Drugs 17: 179-202) it would be expected that exploratory behavioral activity would remain similar among treatment groups, including control rats. Costa et al. (2003) First Eur Workshop on Cannabinoid Research. Madrid (Spain) demonstrated that a CB1 receptor antagonist had no effect on the anti-inflammatory and anti-hyperalgesic effects of CBD. Behavioral changes with CBD treatments were not expected due to the safe nature of the drug compared to the negative side effects associated with THC. THC has been shown to cause hypothermia and hypomobility (Zimmer et al. (1999) Proc Natl Acad Sci USA 96: 5780-5785) which are avoided with the use of CBD. Therefore with CBD treatments, changes particularly in active and resting times would have been observed if CBD's pharmacological actions were similar to THC.

These disclosure presented herein indicate that transdermal CBD has long lasting effect and alleviates the symptoms of arthritis, particularly osteoarthritis.

All references, including publications, patent applications, and patents, cited herein are hereby incorporated by reference to the same extent as if each reference were individually and specifically indicated to be incorporated by reference and were set forth in its entirety herein.

The use of the terms “a,” “an” and “the” and similar references in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the present disclosure.

Alternative embodiments of the claimed disclosure are described herein, including the best mode known to the inventors for practicing the claimed invention. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

We claim:

1. The use of a cannabinoid gel for the preparation of a medicament for the treatment of arthritis, wherein the cannabinoid gel is applied to the skin of a mammal and comprises:
a. a cannabinoid in an amount of about 1% to about 45% (wt/wt) of the composition;
b. a lower alcohol having between 1 and 6 carbon atoms present in an amount of about 15% to about 85% (wt/wt) of the composition;
c. a first penetration enhancer present in an amount of about 0.1% to about 15% (wt/wt) of the composition; and
d. water present in an amount of about 5% to about 45% (wt/wt) of the composition;
wherein, the cannabinoid is selected from the group consisting of: delta-9-tetrahydrocannabinol, delta-8-tetrahydrocannabinol, 6,6,9-trimethyl-3-pentyl-6H-dibenzo[b,d]pyran-1-ol, 3-(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-delta-6-tetrahydrocannabinol-1,1-dimethylheptyl, (+)-(3S,4S)-7-hydroxy-delta-6-tetrahydrocannabinol-1,1-dimethylheptyl, 11-hydroxy-delta-9-tetrahydrocannabinol, delta-8-tetrahydrocannabinol-11-oic acid, (−)-(6S,6aR,9R,10aR)-5,6,6a,7,8,9,10,10a-octahydro-6-methyl-1-3-[(R)-1-methyl-4-phenylbutoxy]-1,9-phenanthridinediol 1-acetate, (R)-(+)-[2,3-dihydro-5-methyl-3-(−4-morpholinylmethyl)-pyrrolo[1,2,3-de]-1,4-benzoxazin-6-yl]-1-naphthalenyl-methanone, 2-[3-methyl-6-(1-methylethenyl)-2-cyclohexen-1-yl]-5-pentyl-1,3-benzenedi-ol and 4-(1,1-dimethylheptyl)-2,3′-dihydroxy-6′ alpha-(3-hydroxypropyl)-1′,-2′,3′,4′,5′,6′-hexahydrobiphenyl, cannabinol, cannabidiol, nabilone, levonantradol, (−)-HU-210, (+)-HU-210,11-hydroxy-delta-9-tetrahydrocannabinol, delta-8-tetrahydrocannabinol-11-oic acid, CP 55,940, and R(+)-WIN 55, 212-2; and
wherein the first penetration enhancer is selected from the group consisting of: N-heptane, N-octane, N-nonane, N-decane, N-undecane, N-dodecane, N-tridecane, N-tetradecane, N-hexadecane, ethanol, propanol, butanol, 2-butanol, pentanol, 2-pentanol, hexanol, octanol, nonanol, decanol, benzyl alcohol, oleyl alcohol, caprylic alcohol, decyl alcohol, lauryl alcohol, 2-lauryl alcohol, myristyl alcohol, cetyl alcohol, stearyl alcohol, oleyl alcohol, linoleyl alcohol, linolenyl alcohol, propylene glycol, polyethylene glycol, ethylene glycol, diethylene glycol, triethylene glycol, dipropylene glycol, glycerol, propanediol, butanediol, pentanediol, hexanetriol, propylene glycol monolaurate, diethylene glycol monomethyl ether (transcutol) urea, dimethylacetamide, diethyltoluamide, dimethylormamide, dimethyloctamide, dimethyldecamide, biodegradable cyclic urea, 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-methyl-pyrrolidone, N-cyclohexylpyrrolidone, N-dimethylaminopropyl-pyrrolidone, N-cocoalkypyrrolidone, N-tallowalkypyrrolidone, esters of N-(2-hydroxyethyl)-2-pyrrolidone, 1-dodecylazacycloheptane-2-one, 1-geranylazacycloheptan-2-one, 1-farnesylazacycloheptan-2-one, 1-geranylgeranylazacycloheptan-2-one, 1-(3,7-dimethyloctyl)-azacycloheptan-2-one, 1-(3,7,11-trimethyldodecyl)azacyclohaptan-2-one, 1-geranylazacyclohexane-2-one, 1-geranylazacyclopentan-2,5-dione, 1-farnesylazacyclopentan-2-one, diethanolamine, triethanolamine, hexamethylenlauramide, octanoic acid, linoleic acid, valeric acid, heptanoic acid, pelagonic acid, caproic acid, capric acid, lauric acid, myristric acid, stearic acid, oleic acid, caprylic acid, isovaleric acid, neopentanoic acid, neoheptanoic acid, neonanoic acid, trimethyl hexaonic acid, neodecanoic acid, isostearic acid, ethyl oleate, isopropyl n-butyrate, isopropyl n-hexanoate, isopropyl n-decanoate, isopropyl myristate (“IPM”), isopropyl palmitate, octyldodecyl myristate, ethyl acetate, butyl acetate, methyl acetate, methylvalerate, methylpropionate, diethyl sebacate, ethyl oleate, butyl stearate, methyl laurate, sodium laurate, sodium lauryl sulfate, sodium octyl sulfate, cetyltrimethylammonium bromide, tetradecyltrimethylammonium, octyltrimethyl ammonium bromide, benzalkonium chloride, octadecyltrimethylammonium chloride, cetylpyridinium chloride, dodecyltrimethylammonium chloride, hexadecyltrimethylammonium chloride, hexadecyl trimethyl ammoniopropane sulfonate, oleyl betaine, cocamidopropyl hydroxysultaine, cocamidopropyl betaine, Polyxamer (231, 182, 184), Polysorbate (20, 60), Brij (30, 93, 96, 99), Span (20, 40, 60, 80, 85), Tween (20, 40, 60, 80), Myrj (45, 51, 52), Miglyol 840, sodium cholate, sodium salts of taurocholic acid, glycolic acids, desoxycholic acids, lecithin, d-limonene, alpha-pinene, beta-carene, alpha-terpineol, terpinen-4-ol, carvol, carvone, pulegone, piperitone, menthone, cyclohexene oxide, limonene oxide, alpha-pinene oxide, cyclopentene oxide, 1,8-cineole, ylang ylang oil, anise oil, chenopodium oil, eucalyptus oil, salicylic acid, salicylates, citric acid succinic acid, 2-hydroxypropyl-beta-cyclodextrin, 2,6-dimethyl-beta-cyclodextrin, alkyl-2-(N,N-disubstituted amino)-alkanoate ester, 2-(n-nonyl)-1,3-dioxolane, diisopropyl adipate, glyceryl monolaurate, tetrahydrofurfuryl alcohol, 2-(2-ethoxyethoxy)ethanol, alkylaryl ethers of polyethylene oxide, polyethylene oxide monomethyl ethers, polyethylene oxide dimethyl ethers, acetoacetic ester, oleoyl macrogolglyceride, caprylocaproyl macrogolylyceride, polyoxyethylene 6 caprylic triglyceride, polyoxyethylene glyceride, PPG-5 ceteth-20, lauroyl macroglyceride oleic acid.

2. The use of claim 1 wherein the cannabinoid is cannabidiol.

3. The use of claim 2, wherein the cannabidiol is present in an amount of about 1% to about 10% (wt/wt) of the composition.

4. The use of claim 2, wherein the cannabidiol is present in an amount of about 1.5% to about 3.5% (wt/wt) of the composition.

5. The use of claim 1, wherein the first penetration enhancer is selected from the group comprises diethylene glycol monoethyl ether or oleyl alcohol.

6. The use of claim 1, wherein the first penetration enhancer is present in an amount of about 0.5% to about 10% (wt/wt) of the composition.

7. The use of claim 1, wherein the first penetration enhancer is diethylene glycol monoethyl ether and is present in an amount of about 7.5% (wt/wt) of the composition.

8. The use of claim 1, wherein the cannabinoid gel further comprises a second penetration enhancer in the amount of about 0.1% to about 5% (wt/wt) of the composition.

9. The use of claim 8, wherein the second penetration enhancer is present in the amount of about 0.1% to about 2% (wt/wt) of the composition.

10. The use of claim 8, wherein the second penetration enhancer is isopropyl myristate.

11. The use of claim 1, wherein the lower alcohol is ethanol or isopropyl alcohol.

12. The use of claim 1, wherein the lower alcohol is present in an amount of about 40% to about 70% (wt/wt) of the composition.

13. The use of claim 1, wherein the lower alcohol is present in an amount of about 45% to about 65% (wt/wt) of the composition.

14. The use of claim 1, wherein water is present in an amount of about 10% to about 40% (wt/wt) of the composition.

15. The use of claim 1, wherein water is present in an amount of about 20% to about 30% (wt/wt) of the composition.

16. The use of claim 1, wherein water is present in an amount of about 25% to about 30% (wt/wt) of the composition.

17. The use of claim 1, wherein the cannabinoid gel further comprises a thickening agent present in an amount of about 0.1% to about 5% (wt/wt) of the composition.

18. The use of claim 17, wherein the thickening agent is selected from the group consisting of: hydroxypropylcellulose, carboxypolymethylene, carboxymethylcellulose, acrylic acid polymer, or neutralized acrylic acid poylmer.

19. The use of claim 17, wherein the thickening agent is partially neutralized polyacrylic acid.

20. The use of claim 17, wherein the thickening agent is present in an amount of about 1% to about 3% (wt/wt) of the composition.

21. The use of claim 1, wherein the composition further comprises a neutralizing agent in an amount of about 0.001% to about 10% (wt/wt) of the composition.

22. The use of claim 21, wherein the neutralizing agent is selected from the group consisting of: triethanolamine, 0.1% aqueous sodium hydroxide solution and 1% aqueous sodium hydroxide solution.

23. The use of claim 21, wherein the neutralizing agent is triethanolamine and is present in amount of about 0.1% to about 0.2% (wt/wt of the composition.

24. The use of claim 1, wherein the cannabinoid gel further comprises a first antioxidant, present in the amount of about 0.01% to about 1% (wt/wt) of the composition.

25. The use of claim 24, wherein the first antioxidant is selected from the group consisting of: citric acid, butylated hydroxytoluene, ascorbic acid, glutathione, retinol, α-tocopherol, β-carotene, α-carotene, ubiquinone, butylated hydroxyanisole, ethylenediaminetetraacetic acid, selenium, zinc, lignan, uric acid, lipoic acid, and N-acetylcysteine.

26. The use of claim 1, wherein the cannabinoid gel further comprises a second antioxidant present in an amount of about 0.01% to about 1% (wt/wt) of the composition.

27. The use of claim 26, wherein the second antioxidant is selected from the group consisting of: citric acid, butylated hydroxytoluene, ascorbic acid, glutathione, retinol, α-tocopherol, β-carotene, α-carotene, ubiquinone, butylated hydroxyanisole, ethylenediaminetetraacetic acid, selenium, zinc, lignan, uric acid, lipoic acid, and N-acetylcysteine.

28. The use of claim 1, where in the cannabinoid gel further comprises propylene glycol present in an amount of about 1% to about 25% (wt/wt) of the composition.

29. The use of claim 28, wherein the propylene glycol is present in an amount of about 1% to about 20% (wt/wt) of the composition.
